(19) United States
(12) Patent Application Publication
(10) Pub. No.: US 2012/0108461 A1
(43) Pub. Date: May 3, 2012

(54) HIGH-THROUGHPUT SLIDE PROCESSING APPARATUS

(75) Inventors: John E. Bussan, Naperville, IL (US); Michael R. Nelson, Libertyville, IL (US); Pat N. Morgan, Highland Park, IL (US); Sergey V. Rozhok, Skokie, IL (US); Jeff Rendlin, Glen Ellyn, IL (US)

(73) Assignee: Nanolnk, Inc.

(21) Appl. No.: 13/286,078

(22) Filed: Oct. 31, 2011

Related U.S. Application Data
(60) Provisional application No. 61/409,070, filed on Nov. 1, 2010.

(51) Int. Cl. C40B 30/04; G01N 35/00; C40B 40/10; G01N 33/543

(52) U.S. Cl. 506/9; 422/67; 436/518; 436/46; 506/18

(57) ABSTRACT

An assay device and method of use thereof includes a sample tray comprising a plurality of sample wells having a first volume, and a slide tray comprising a slide, and a liquid dispenser. The slide comprises a plurality of reaction sites on a bottom surface of the slide. The liquid dispenser is configured to dispense a plurality of liquid samples into the sample wells. The sample wells are configured to hold the liquid samples. Each of the liquid samples has a second volume such that the second volume exceeds the first volume and each of the liquid sample sits within and above one of the sample wells. The slide tray and the sample tray are configured such that the slide tray can be placed onto the sample tray, at least one of the reaction sites can be positioned directly above at least one of the sample wells containing a liquid sample, and the liquid sample can be drawn onto the reaction site upon the liquid sample contacting the bottom surface of the slide.

### RELATED APPLICATION

This application claims priority to U.S. provisional application 61/409,070, which is hereby incorporated by reference in its entirety.

### BACKGROUND

To meet the challenges of new genomics and proteomic applications, it is required to achieve accurate and efficient analysis of large numbers of interactions simultaneously. High-density microarrays are ideally suited for concurrent multiplex screening of thousands of interactions with minimal use of materials. Most (if not all) technologies allowing detection and screening of multiple biological analytes in vitro use a solid phase platform like glass slides, membranes, microliter wells, mass spectrometer plates, beads, or other particles in order to build arrays of multiple sites for capturing target molecules from solution. The prior art glass slide platforms for running biological assay are generally limited to 16 or 24 microarrays per slide. Some academic groups have demonstrated developments in which 48- or 96-well arrays on a structured single glass slide were printed and hybridized with one sample per array. See Huang et al., Clinical Chemistry, 47(10):1912-1916 (2001).

Development of microarray printing technology allows the simultaneously parallel printing of molecules on a small area, which allows the measurement of considerable number of molecular interactions in a single experiment. However, to achieve all benefits of high throughput printing and running assays on a micron scale, it is necessary to provide access of liquid reagents to extremely small areas with no cross contamination to surrounding. The typical format for running assay on a glass slide uses gaskets that localize each array to a single reaction well. However gaskets can not conform to very small wells because high surface tension of liquid reagents to the gasket material prevents them from reaching the glass surface. In addition, the number of assay wells that can be created on a single slide is limited by the minimum required gasket wall thickness.

Automated processing equipment allows for a significant increase in capability, flexibility, and speed in the handing of liquid reagents. Prior art devices required manual assembly of a printed slide sandwiched between plastic parts with an elastomer gasket forming an assay well at each printed sample. The printed slide faces up and assay liquids are added manually or automatically using pipettes. The liquid then must be poured off and the process repeated, often multiple times. This requires substantial manual labor, is time-consuming and is prone to error. To achieve all benefits of high throughput printing and allow accurate and efficient high-throughput assay, it is desirable to use an automated workstation. Microscope slide handling systems are not readily available. The slide processing equipment that does exist is specialized to very specific tasks, such as fluorescent microscopy. Such equipment cannot be adapted for generic handing of liquid reagents.

Unlike microscope slide handling systems, automated titer plate laboratory workstations are readily available.

### SUMMARY

Embodiments described herein include, for example, methods of making, methods of using, and devices.

For example, one embodiment provides a method comprising: providing an automation workstation comprising a gripper, a liquid dispenser, and a plurality of workstation positions, providing a sample tray in a first workstation position, wherein the sample tray comprises a plurality of sample wells having a first volume, providing a slide tray comprising at least one slide, wherein the slide comprises a plurality of reaction sites on a bottom surface of the slide, depositing a liquid sample having a second volume into at least one of the sample wells using the liquid dispenser such that the second volume exceeds the first volume and the liquid sample sits within and above one of the sample wells, moving the slide tray to the first workstation position using the gripper, and placing the slide tray onto the sample tray using the gripper such that at least one of the reaction sites is positioned directly above at least one of the sample wells containing a liquid sample and the liquid sample is drawn onto the reaction site upon the liquid sample contacting the bottom surface of the slide. Optionally, the slide tray can comprise at least two, or at least three, slides.

One embodiment further comprises providing a bath tray in a second workstation position, wherein the bath tray comprises a bath well, moving the slide tray to the second workstation position using the gripper, placing the slide tray onto the bath tray using the gripper, and depositing a bath liquid into the bath well using the liquid dispenser such that the bath liquid contacts the plurality of reaction sites.

Another embodiment comprises providing a wash tray in a third workstation position, wherein the wash tray comprises a wash well, moving the slide tray to the third workstation position using the gripper, placing the slide tray onto the wash tray using the gripper, and depositing a wash buffer into the wash well such that the wash buffer contacts the plurality of reaction sites. In another embodiment, the wash buffer is deposited using a liquid dispenser. In another embodiment, the wash buffer is deposited using a second liquid dispenser. In another embodiment, the wash tray is permanently attached at the third workstation position. In another embodiment, the wash tray can be moved using the gripper. Another embodiment comprises the step of placing a weight on the slides in the slide tray. Another embodiment comprises the step of using a vacuum device to create a vacuum in the space between the slide and the bath tray.

One embodiment further comprises the steps of providing a bath tray in a second workstation position, wherein the bath tray comprises a bath well, moving the slide tray to the second workstation position using the gripper, placing the slide tray onto the bath tray using the gripper, and depositing a bath liquid into the bath well using the liquid dispenser such that the bath liquid contacts the plurality of reaction sites.

One embodiment further comprises the steps of providing a wash tray in a third workstation position, wherein the wash tray comprises a wash well, moving the slide tray to the third workstation position using the gripper, placing the slide tray onto the wash tray using the gripper, and depositing a wash buffer into the wash well such that the wash buffer contacts the plurality of reaction sites.

In one embodiment, the wash buffer is deposited using the liquid dispenser. In one embodiment, the wash buffer is deposited using a second liquid dispenser.

In one embodiment, the wash tray is permanently attached at the third workstation position. In one embodiment, the wash tray can be moved using the gripper.

One embodiment further comprises the step of placing a weight on the slides in the slide tray. One embodiment further comprises the step of using a vacuum device to create a vacuum in the space between the slide and the bath tray.

In one embodiment, the slide trays are stackable. In one embodiment, the slide tray is moved to the first workstation position from a slide tray input stack using the gripper. One embodiment further comprises the step of moving the slide tray to a slide tray output stack using the gripper.

In one embodiment, the sample trays are stackable. One embodiment further comprises the step of moving the sample tray to the first workstation position from a sample tray input stack using the gripper. One embodiment further comprises the step of moving the sample tray to a sample tray output stack using the gripper.

In one embodiment, the slide tray comprises at least one additional slide.

In one embodiment, the sample tray is made of plastic. In one embodiment, the sample tray is made of a solid piece of plastic. In one embodiment, the sample tray is of rectangular shape.

In one embodiment, the number of sample wells is selected from the group consisting of 48, 96, and 384.

In one embodiment, the distance between neighboring sample wells is about 4.5 mm.

In one embodiment, the sample wells are round.

In one embodiment, the depth of the sample wells is less that 500 μm. In one embodiment, the depth of the sample wells is less than 300 μm. In one embodiment, the depth of the sample wells is less than 160 μm.

In one embodiment, the liquid dispenser comprises a pipette. In one embodiment, the first volume is less than 2.5 μl. In one embodiment, the first volume is less than 1 μl.

In one embodiment, the second volume is selected from a group consisting of 4 μl, 2.5 μl, or 1 μl.

In one embodiment, the distance from the bottom of the sample well to the top of the liquid sample when the liquid sample is in the well is greater than the distance between the bottom of the sample well and the bottom surface of the slide when the slide tray is placed on the sample tray.

In one embodiment, the liquid sample comprises analytes capable of being captured by the reaction sites and the reaction sites comprise capture molecules capable of capturing analytes.

In one embodiment, the liquid sample comprises antigens and the reaction site comprises antibodies.

In one embodiment, the automation workstation is a titer plate laboratory workstation. In one embodiment, the slide comprises a glass material. In one embodiment, the slide comprises a solid piece of epoxy glass.

In one embodiment, the reaction sites comprise antibodies.

In one embodiment, the reaction sites are printed onto the slide via a Dip Pen Nanolithography process.

In one embodiment, the positions of the reaction sites match the positions of the sample wells.

In one embodiment, the bottom surface of the slide comprises a hydrophilic material.

In one embodiment, the liquid sample creates a reaction volume over one of the reaction sites upon contacting the bottom surface of the slide.

One embodiment further comprises the step of securing the slide to the slide tray. In one embodiment, the slide is secured to the slide tray using a screw.

Another embodiment provides an article comprising: an automation workstation comprising a gripper, a liquid dispenser, and a plurality of workstation positions, a sample tray configured to be placed in a first workstation position, wherein the sample tray comprises a plurality of sample wells having a first volume, a slide tray comprising a slide, wherein the slide comprises a plurality of reaction sites on a bottom surface of the slide, wherein the liquid dispenser is configured to dispense a plurality of liquid samples into the sample wells, wherein the sample wells are configured to hold the liquid samples, each of the liquid samples having a second volume such that the second volume exceeds the first volume and each of the liquid sample sits within and above one of the sample wells, wherein the slide tray is configured to be movable to the first workstation position using the gripper, and wherein the slide tray and the sample tray are configured such that the slide tray can be placed onto the sample tray using the gripper, at least one of the reaction sites can be positioned directly above at least one of the sample wells containing a liquid sample, and the liquid sample can be drawn onto the reaction site upon the liquid sample contacting the bottom surface of the slide.

One embodiment further comprises: a bath tray configured to be placed in a second workstation position, wherein the bath tray comprises a bath well, wherein the slide tray is configured to be movable to the second workstation position using the gripper, and wherein the slide tray and the bath tray are configured such that the slide tray can be placed on the bath tray using the gripper and bath liquid can be deposited into the bath well such that the bath liquid contacts the plurality of reaction sites.

One embodiment further comprises: a wash tray configured to be placed in a third workstation position, wherein the wash tray comprises a wash well, wherein the wash tray is configured to be movable to the third workstation position using the gripper, and wherein the slide tray and the wash tray are configured such that the slide tray can be placed on the wash tray using the gripper and wash buffer can be deposited into the wash well such that the wash buffer contacts the plurality of reaction sites.

In one embodiment, the liquid dispenser of claim 43 is configured to deposit the wash buffer. One embodiment further comprises a second liquid dispenser that is configured to deposit the wash buffer.

In one embodiment, the wash tray is permanently attached at the third workstation position. In one embodiment, the wash tray is configured to be movable using the gripper.

One embodiment further comprises a weight configured to be placed on the slides in the slide tray.

One embodiment further comprises a vacuum device configured to create a vacuum in the space between the slide and the bath tray.

In one embodiment, the slide trays are stackable. In one embodiment, the slide tray is configured to be movable to the first workstation position from a slide tray input stack using the gripper. In one embodiment, the slide tray is configured to be movable to a slide tray output stack using the gripper.

In one embodiment, the sample trays are stackable. In one embodiment, the sample tray is configured to be movable to the first workstation position from a sample tray input stack using the gripper. In one embodiment, the sample tray is configured to be movable to a sample tray output stack using the gripper.

In one embodiment, the slide tray comprises at least one additional slide.

In one embodiment, the sample tray is made of plastic. In one embodiment, the sample tray is made of a solid piece of plastic. In one embodiment, the sample tray is of rectangular shape.

In one embodiment, the number of sample wells is selected from the group consisting of 48, 96, and 384.

In one embodiment, the distance between neighboring sample wells is about 4.5 mm.

In one embodiment, the sample wells are round.

In one embodiment, the depth of the sample wells is less that 500 μm. In one embodiment, the depth of the sample wells is less than 300 μm. In one embodiment, the depth of the sample wells is less than 160 μm.

In one embodiment, the liquid dispenser comprises a pipette.

In one embodiment, the first volume is less than 2.5 μl. In one embodiment, the first volume is less than 1 μl.

In one embodiment, the second volume is selected from a group consisting of 4 μl, 2.5 μl, or 1 μl.

In one embodiment, the distance from the bottom of the sample well to the top of the liquid sample when the liquid sample is in the well is greater than the distance between the bottom of the sample well and the bottom surface of the slide when the slide tray is placed on the sample tray.

In one embodiment, the liquid sample comprises analytes capable of being captured by the reaction site and the reaction sites comprise capture molecules capable of capturing analytes.

In one embodiment, the liquid sample comprises antigens and the reaction site comprises antibodies.

In one embodiment, the automation workstation is a titer plate laboratory workstation.

In one embodiment, the slide comprises a glass material. In one embodiment, the slide comprises a solid piece of epoxy glass. In one embodiment, the reaction sites comprise antibodies.

In one embodiment, the reaction sites are printed onto the slide via a Dip Pen Nanolithography process.

In one embodiment, the positions of the reaction sites match the positions of the sample wells.

In one embodiment, the bottom surface of the slide comprises a hydrophilic material.

In one embodiment, the liquid sample creates a reaction volume over one of the reaction sites upon contacting the bottom surface of the slide.

One embodiment further comprises a fastener configured to secure the slide to the slide tray. In one embodiment, the fastener is a screw.

Another embodiment provides an article comprising a sample tray comprising a plurality of sample wells having a first volume, a slide tray comprising a slide, wherein the slide comprises a plurality of reaction sites on a bottom surface of the slide, and a liquid dispenser configured to dispense a plurality of liquid samples into the sample wells. The sample wells are configured to hold the liquid samples, each of the liquid samples having a second volume such that the second volume exceeds the first volume and each of the liquid sample sits within and above one of the sample wells. The slide tray and the sample tray are configured such that the slide tray can be placed onto the sample tray, at least one of the reaction sites can be positioned directly above at least one of the sample wells containing a liquid sample, and the liquid sample can be drawn onto the reaction site upon the liquid sample contacting the bottom surface of the slide.

In one embodiment, the sample tray has outside dimensions that are substantially similar to the outside dimensions of a standard titer plate and the slide tray has outside dimensions that are substantially similar to the outside dimensions of a standard titer plate.

At least one advantage for at least one embodiment includes the capability of sealing the liquid sample between the slide tray and the sample tray or bath tray, which prevents the liquid sample from evaporating and prevents outside contamination, while allowing long incubation times.

At least one advantage for at least one embodiment includes keeping liquid samples within an extremely small area on a slide with no structural modifications to eliminate cross contamination with surrounding samples.

At least one advantage for at least one embodiment includes eliminating the need to make, use, or clean a gasket.

At least one advantage for at least one embodiment includes minimum use of samples and reaction sites while generating a large amount of data.

At least one advantage for at least one embodiment includes the capability for massive concurrent quantitative measurements with high reliability.

At least one advantage for at least one embodiment includes ease of use in automated applications.

At least one advantage for at least one embodiment includes low production cost of the slide tray, sample tray, and wash tray.

At least one advantage for at least one embodiment includes the ability to use commonly available laboratory automation equipment in order to process slides accurately and efficiently.

At least one advantage of at least one embodiment includes the ability to use a titer plate laboratory workstation in order to process slides accurately and efficiently.

### BRIEF DESCRIPTION OF THE DRAWINGS

FIG. 1 illustrates an exploded perspective view of known device used for liquid assay of a slide.

FIG. 2 illustrates a perspective view of a device used for liquid assay of a slide on a chip with a number of wells.

FIG. 3 illustrates an exploded perspective view of a device used for liquid assay of a slide.

FIG. 4 illustrates a perspective view of the device illustrated in FIG. 3.

FIG. 5 illustrates a front perspective view of an automation workstation that can be used in the present invention.

FIG. 6 illustrates a top perspective view of a standard ANSI/SBS format titer plate.

FIG. 7 illustrates a top view of a standard ANSI/SBS format titer plate.

FIG. 8 illustrates a side perspective view of a gripper of the automation workstation illustrated in FIG. 5, shown here moving a titer plate.

FIG. 9 illustrates a perspective view of a liquid dispenser of automation workstation illustrated in FIG. 5, shown here with a number of pipettes.

FIG. 10 illustrates a number of slide trays, sample trays, and bath trays located at located at various workstation positions on the deck of the automation workstation illustrated in FIG. 5.

FIG. 11 illustrates a top perspective view of a sample tray with a number of wells according to an embodiment of the present invention.

FIG. 12 illustrates a top view of two possible configurations of the wells on the sample tray illustrated in FIG. 11.

FIG. 13 illustrates a top view of three possible configurations of the wells on the sample tray illustrated in FIG. 11.

FIG. 14 illustrates a perspective view of a stack of the sample trays illustrated in FIG. 11.

FIG. 15 illustrates a perspective view of a slide tray according to an embodiment of the present invention.

FIG. 16 illustrates a perspective view of a slide tray with a slide according to an embodiment of the present invention.

FIG. 17 illustrates a perspective view of a stack of slide trays, each with a slide, according to an embodiment of the present invention.

FIG. 18 illustrates a perspective view of a slide with a number of reaction sites.

FIG. 19 illustrates a top perspective view of a possible array of several different types of reaction sites on a slide.

FIG. 20 illustrates an exploded perspective view of the slide tray and slide illustrated in FIG. 16, shown here with a number of weights, according to an embodiment of the present invention.

FIG. 21 illustrates an top perspective view of a slide tray, slide, and weights, according to an embodiment of the present invention.

FIG. 22 illustrates a sample well on a sample tray, shown here with a liquid sample in the sample well, according to an embodiment of the present invention.

FIG. 23 illustrates a front cross-sectional view of the structure of the sample tray, the shape of the liquid samples sitting in the wells, the placement of the slide, and the transformation of the shape of liquid samples upon contacting the slide, according to an embodiment of the present invention.

FIG. 24 illustrates a top perspective view of a bath tray with a reservoir and vacuum ports, according to an embodiment of the present invention.

FIG. 25 illustrates a top perspective view of a bath tray with a series of slots, according to an embodiment of the present invention.

FIG. 26 illustrates a top perspective view of a bath tray according to an embodiment of the present invention.

FIG. 27 illustrates a top perspective view of a stack of bath trays, according to an embodiment of the present invention.

FIG. 28 illustrates a top perspective view of a bath try on a wash station, according to an embodiment of the present invention.

FIG. 29 illustrates a perspective view of a mount on which a slide tray, sample tray, bath tray, or wash tray can be placed in a workstation position.

FIG. 30 illustrates a bottom perspective view of a slide tray with a slide.

FIG. 31 illustrates an exploded perspective view of an embodiment in which a machined sample slide insert is mounted to a sample tray frame using a fastener to form a sample tray.

FIG. 32 illustrates an exploded perspective view of an embodiment in which a machined sample slide insert is mounted to a sample tray frame using a block that holds the sample slide insert and is mounted to the sample tray frame using a fastener to form a sample tray.

FIG. 33 illustrates a sample tray made of a layer of non-hydrophobic material and a layer of hydrophobic material disposed over the layer of non-hydrophobic material, along with a slide tray and an insert.

FIG. 34 illustrates a slide tray and an insert that can be removably disposed in the slide tray.

FIG. 35A illustrates nozzle that can be used as a part of a washing station in certain embodiments.

FIG. 35B illustrates a close-up of the nozzle of FIG. 35, showing a fluid slot and a vacuum slot.

FIG. 36 illustrates a washing station with a slide tray disposed thereon.

FIG. 37 illustrates a syringe pump that can be used in certain embodiments.

FIG. 38 illustrates a “peripheral vacuum” drying station according to one embodiment.

### DETAILED DESCRIPTION

### Introduction

Priority U.S. provisional application 61/409,070 is hereby incorporated by reference in its entirety.

U.S. provisional application entitled “High-Throughput Assay Methods and Articles” to Rozhok et al., assigned to NanoInk, Inc., Ser. No. 61/409062, filed Nov. 1, 2010, is incorporated herein by reference in its entirety. This application describes unit operations which can be adapted for use with the work station embodiments described herein. Cofiled application Ser. No. ______, assignee: NanoInk, Inc., “High-Throughput Assay Methods and Articles,” is also incorporated by reference in its entirety including the claims and supporting application text. For example, embodiments described therein include:

Embodiment 1. A method comprising providing a chip comprising a top surface, edges surrounding the top surface, a plurality of wells of a first volume on the top surface, and, optionally, shoulders along the edges and elevated from the top surface; providing a slide comprising a bottom surface and at least one reactive site on the bottom surface; administering at least one liquid sample of a second volume into at least one of the wells, wherein the second volume exceeds the first volume, and wherein the liquid sample sits within and above the well; and placing the slide over the chip such that the reactive site is positioned above at least one of the wells and contacts the liquid sample.

Embodiment 2. The method of embodiment 1, wherein the shoulder is not optional but present, and the placing of the slide results in the slide contacting the shoulder.

Embodiment 3. The method of embodiment 1, wherein the optional shoulder is not present.

Embodiment 4. The method of embodiment 1, wherein the chip is made of plastic.

Embodiment 5. The method of embodiment 1, wherein the number of wells is at least 24.

Embodiment 6. The method of embodiment 1, wherein the number of wells is at least 96.

Embodiment 7. The method of embodiment 5, wherein the wells are disposed on the top surface in a regular array layout.

Embodiment 8. The method of embodiment 1, wherein the distance between the wells matches the pitch between the tips of multichannel pipettes or liquid handling systems.

Embodiment 9. The method of embodiment 1, wherein the distance between neighboring wells is about 2.5 mm to about 9 mm.

Embodiment 10. The method of embodiment 1, wherein the well is of round shape.

Embodiment 11. The method of embodiment 1, wherein wells of the chip are formed from a patterned layer formed on a substrate.

Embodiment 12. The method of embodiment 1, wherein the depth of the well is about 25 microns to about 500 microns.

Embodiment 13. The method of embodiment 1, wherein the depth of the well is about 100 microns to about 250 microns.

Embodiment 14. The method of embodiment 1, wherein the depth of the well is about 140 microns to about 180 microns.

Embodiment 15. The method of embodiment 1, wherein the first volume is less than 2.5 ul.

Embodiment 16. The method of embodiment 1, wherein the first volume is less than 1 ul.

Embodiment 17. The method of embodiment 1, wherein the shoulder is present and the height of the shoulder is about one mm or less.

Embodiment 18. The method of embodiment 1, wherein the shoulder is present and the height of the shoulder is about 650 microns or less.

Embodiment 19. The method of embodiment 1, wherein the liquid sample is administered manually through multichannel pipettes.

Embodiment 20. The method of embodiment 1, wherein the liquid sample is administered through an automated liquid handling system.

Embodiment 21. The method of embodiment 1, wherein the second volume is about 0.5 microliters to about 25 microliters.

Embodiment 22. The method of embodiment 1, wherein the liquid sample sits in the well in a hemisphere shape.

Embodiment 23. The method of embodiment 1, wherein the shoulder is present and the distance from the bottom of the well to the top of the liquid sample sitting in the well exceeds the depth of the well plus the height of the shoulder.

Embodiment 24. The method of embodiment 1, wherein the liquid sample comprises analytes capable of being captured by the reactive site.

Embodiment 25. The method of embodiment 1, wherein the liquid sample comprises antigens and wherein the reactive sites comprises antibodies.

Embodiment 26. The method of embodiment 1, wherein the slide is made of glass.

Embodiment 27. The method of embodiment 1, wherein the slide is a solid piece of epoxy glass.

Embodiment 28. The method of embodiment 1, wherein the slide is a solid piece of epoxy glass printed with an array of antibodies for reactive sites.

Embodiment 29. The method of embodiment 1, wherein the reaction site is printed onto the slide via Dip Pen Nanolithography process.

Embodiment 30. The method of embodiment 1, wherein the reaction site is printed with use of direct write nanolithography.

Embodiment 31. The method of embodiment 1, wherein the reaction site is printed with use of a stamping process or a non-contact printing process.

Embodiment 32. The method of embodiment 1, wherein the positions of the reaction site matches the positions of the wells.

Embodiment 33. The method of embodiment 1, wherein the reaction site comprises at least one capture molecule capable of capturing analytes.

Embodiment 34. The method of embodiment 1, wherein the bottom surface of the slide is hydrophilic.

Embodiment 35. The method of embodiment 1, wherein the liquid sample transforms to a cylindrical shape upon contacting the bottom surface of the slide.

Embodiment 36. The method of embodiment 1, wherein the liquid sample creates a reaction volume over the reactive site upon contacting the bottom surface of the slide.

Embodiment 37. The method of embodiment 1, wherein the shoulder is present and placement of the slide on the shoulder creates a closed incubation chamber preventing the liquid samples from evaporation and outside contamination.

Embodiment 38. The method of embodiment 1, further comprising the step of securing the slide to the chip.

Embodiment 39. The method of embodiment 1, wherein the slide is secured to the chip using a weight or with a screw.

Embodiment 40. The method of embodiment 1, wherein the method is carried out without use of a gasket.

Embodiment 41. A method comprising providing a chip comprising a first surface comprising a plurality of wells of a first volume on the first surface; providing a slide comprising a first surface and at least one array of reactive sites on the first surface; disposing at least one liquid sample of a second volume into at least one of the wells, wherein the second volume substantially exceeds the first volume, and wherein the liquid sample sits within and above the well; contacting the liquid sample with the array of reactive site, wherein a gasket is not used to surround the liquid sample.

Embodiment 42. The method of embodiment 41, wherein the contacting step is carried out so that the chip and the slide are separated by a predetermined distance.

Embodiment 43. The method of embodiment 41, wherein the array is printed on the slide by a direct write nanolithographic process.

Embodiment 44. The method of embodiment 41, wherein the contacting step is carried out so that the chip and the slide are separated by a predetermined distance determined by a height of a shoulder disposed on the chip.

Embodiment 45. The method of embodiment 41, wherein the number of wells is at least 48 and the number of reaction sites in the array is at least 48.

Embodiment 46. The method of embodiment 41, wherein the reaction sites are separated from each other in the array by about 10 nm to about 100 microns.

Embodiment 47. The method of embodiment 41, wherein the second volume is about 0.5 microliters to about 25 microliters.

Embodiment 48. The method of embodiment 41, wherein the well has an average well depth of about 25 microns to about 500 microns.

Embodiment 49. The method of embodiment 41, wherein the well has an average well diameter of about 1 mm to about 5 mm.

Embodiment 50. The method of embodiment 41, wherein the contact results in a compression of the droplet.

Embodiment 51. An article, comprises: a chip defining a top surface and edges surrounding the top surfaces, having at least one well on the top surface for receiving liquid, and comprising, optionally, a shoulder along the edges and elevated from the top surface; a slide disposed on the chip and defining a bottom surface and comprising at least one reaction site on the bottom surface aligned opposite of the well.

Embodiment 52. The article of embodiment 51, wherein the optional shoulder is present, and the slide is detachably placed on the shoulders for contacting and drawing liquid from the well onto the reactive site.

Embodiment 53. The article of embodiment 51, wherein the chip is made of plastic.

Embodiment 54. The article of embodiment 51, wherein the chip is a solid piece of plastic of rectangular shape with machined top surface.

Embodiment 55. The article of embodiment 51, wherein the number of wells is at least 48.

Embodiment 56. The article of embodiment 51, wherein the wells are disposed on the top surface in an array layout.

Embodiment 57. The article of embodiment 51, wherein the distance between the wells matches the pitch between the tips of commercially available multichannel pipettes or liquid handling systems.

Embodiment 58. The article of embodiment 51, wherein the well is of round shape.

Embodiment 59. The article of embodiment 51, wherein the depth of the well is less than 500 um.

Embodiment 60. The article of embodiment 51, wherein the depth of the well is less than 300 um.

Embodiment 61. The article of embodiment 51, wherein the depth of the well is less than 160 um.

Embodiment 62. The article of embodiment 51, wherein the volume of the well is less than 2.5 ul.

Embodiment 63. The article of embodiment 51, wherein the volume of the well is less than 1 ul.

Embodiment 64. The article of embodiment 51, wherein the shoulder is present and the height of the shoulder is no more than 450 um.

Embodiment 65. The article of embodiment 51, wherein the shoulder is present and the height of the shoulder is no more than 200 um.

Embodiment 66. The article of embodiment 51, wherein the slide is made of glass.

Embodiment 67. The article of embodiment 51, wherein the slide is a solid piece of epoxy glass.

Embodiment 68. The article of embodiment 51, wherein the slide is a solid piece of epoxy glass printed with an array of antibodies to form the reaction sites.

Embodiment 69. The article of embodiment 51, wherein the reaction site is printed onto the slide via Dip Pen Nanolithography process.

Embodiment 70. The article of embodiment 51, wherein the position of the reaction site matches the position of the well.

Embodiment 71. The article of embodiment 51, wherein the reaction site comprises capture molecules capable of capturing one or more analytes.

Embodiment 72. The article of embodiment 51, wherein the bottom surface of the slide is hydrophilic.

Embodiment 73. The article of embodiment 51, wherein the placement of the slide on the shoulders create a closed incubation chamber preventing both outside contamination and liquid evaporation.

Embodiment 74. The article of embodiment 51, further comprising a weight being placed on the slide for securing the slide on the chip.

Embodiment 75. The article of embodiment 51, further comprising a screw for securing the slide on the chip.

Embodiment 76. An article comprising: a chip of rectangular shape made of plastic, said chip comprising a top surface being machined, edges surrounding the top surfaces, a plurality of wells on the top surface for receiving liquid, and shoulders along the edges and elevated from the top surface; a slide made of epoxy glass, said slide comprising a bottom surface of hydrophilic nature and a plurality of capture molecules on the bottom surface; wherein the depth of the well is no more than 160 um, the volume of the well is no more than 1 ul, the height of the shoulder is no more than 450 um, the number of the wells is selected from the group consisting of 48, 96, 384, and the distance between the wells matches the pitch between the tips of commercially available multichannel pipettes or liquid handling systems; wherein the capture molecules is printed on the bottom surface via a direct write nanolithography process, the capture molecules are capable of capturing at least one analyte from a liquid sample, and the position of the capture molecules matches the position of the wells; and wherein the slide is detachably placed on the shoulders, is capable of contacting and drawing liquid from the well onto the capture molecules, and is capable of creating a closed incubation chamber preventing both outside contamination and liquid evaporation.

Embodiment 77. A method comprising providing a chip comprising a first surface comprising a plurality of wells of a first volume on the first surface; providing a slide comprising a first surface and at least one array of reactive sites on the first surface; disposing bulk liquid over the wells, and; contacting the bulk liquid with the array of reactive sites.

Additional embodiments are described in cofiled application Ser. No. ______, assignee: Nanolnk, Inc., “High-Throughput Assay Methods and Articles” including those illustrated in the figures.

Printing based on nanoscopic tips is described in, for example, U.S. Pat. Nos. 6,635,311; 6,827,979; 7,361,310; 7,569,340; 7,722,928; and patent publication nos. 2003/0068446 and 2005/0009206, as well as WO/2009/132321 published Oct. 29, 2009 (assignee: Northwestern University), which are hereby incorporated by reference. These methods can be used to prepare microarrays and print assays or reactive sites. Other printing methods such as stamping and direct write lithography are known.

Microarrays are generally known in the art. See, e.g., Kohane, Kho, and Butte, Microarrays for an Integrative Genomics, 2003; and Müller, Roder, Microarrays, 2006. For example, the Muller text describes protein microarrays, nucleic acid microarrays, microarray detection, and microarray marking systems. It also describes microarray spotters, microarray scanners and digitizing, microarray software and documentation, additional laboratory equipment, and clean room technology. All references cited herein are incorporated by reference in their entirety.

FIG. 1 depicts a known device used for liquid assay of a slide. The device uses a gasket with a series of wells. In embodiments described herein, this gasket can be eliminated.

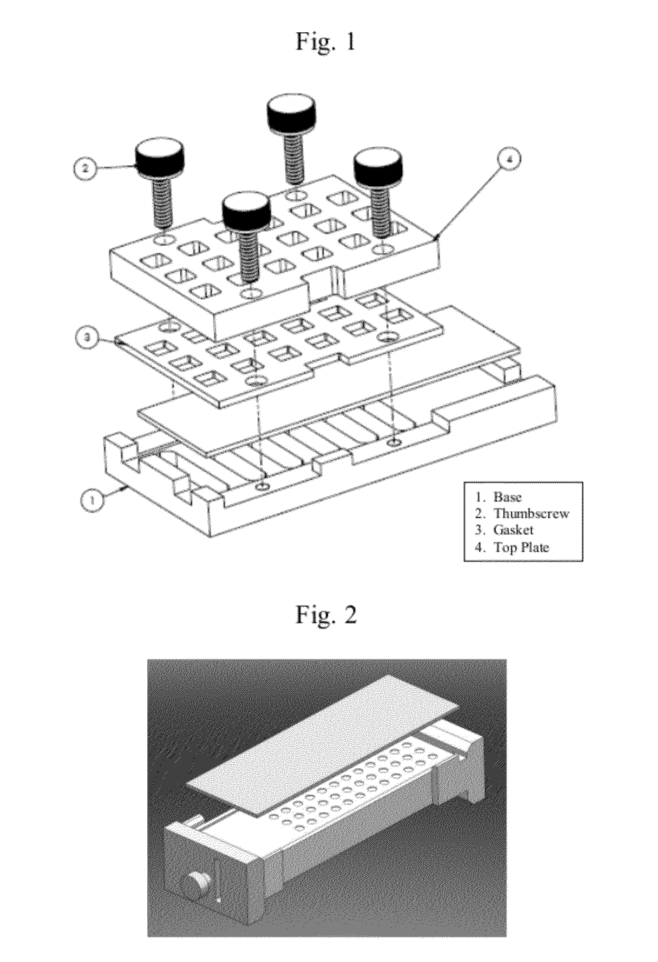

FIG. 2 depicts a device used for liquid assay of a slide on a chip with a number of wells.

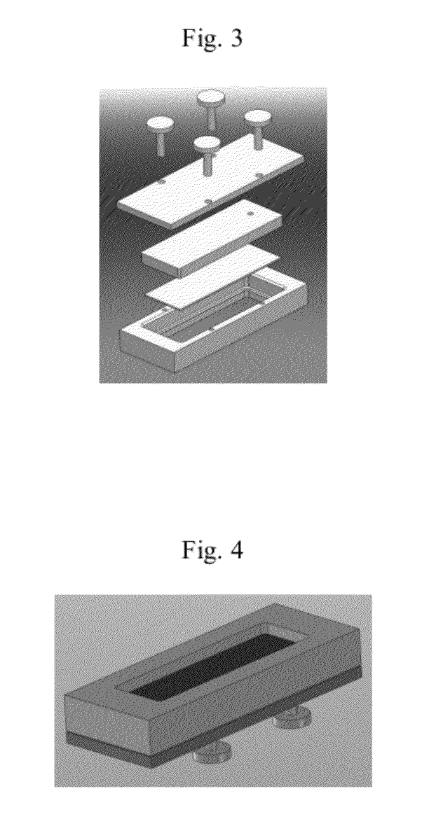

FIGS. 3 and 4 illustrates a bath tray used to expose a slide to bulk quantities of assay liquids. The slide is sealed against the frame by assembling the tray as shown in FIG. 3, with the printed array side down. The bath tray is then turned over so that the printed side faces up, as in FIG. 4. Assay liquids and wash/buffer liquids are added and removed multiple time to complete the assay. This bath tray is also used for washing/buffering the slides.

One embodiment of the present invention provides a method comprising: an automation workstation comprising a gripper, a liquid dispenser, and a plurality of workstation positions, providing a sample tray in a first workstation position, wherein the sample tray comprises a plurality of sample wells having a first volume, providing a slide tray comprising at least one slide, wherein the slide comprises a plurality of reaction sites on a bottom surface of the slide, depositing a liquid sample having a second volume into at least one of the sample wells using the liquid dispenser such that the second volume exceeds the first volume and the liquid sample sits within and above one of the sample wells, moving the slide tray to the first workstation position using the gripper, and placing the slide tray onto the sample tray using the gripper such that at least one of the reaction sites is positioned directly above at least one of the sample wells containing a liquid sample and the liquid sample is drawn onto the reaction site upon the liquid sample contacting the bottom surface of the slide.

Another embodiment provides an article comprising: an automation workstation comprising a gripper, a liquid dispenser, and a plurality of workstation positions, a sample tray configured to be placed in a first workstation position, wherein the sample tray comprises a plurality of sample wells having a first volume, a slide tray comprising a slide, wherein the slide comprises a plurality of reaction sites on a bottom surface of the slide, wherein the liquid dispenser is configured to dispense a plurality of liquid samples into the sample wells, wherein the sample wells are configured to hold the liquid samples, each of the liquid samples having a second volume such that the second volume exceeds the first volume and each of the liquid sample sits within and above one of the sample wells, wherein the slide tray is configured to be movable to the first workstation position using the gripper, and wherein the slide tray and the sample tray are configured such that the slide tray can be placed onto the sample tray using the gripper, at least one of the reaction sites can be positioned directly above at least one of the sample wells containing a liquid sample, and the liquid sample can be drawn onto the reaction site upon the liquid sample contacting the bottom surface of the slide.

Another embodiment provides an article comprising a sample tray comprising a plurality of sample wells having a first volume, a slide tray comprising a slide, wherein the slide comprises a plurality of reaction sites on a bottom surface of the slide, and a liquid dispenser configured to dispense a plurality of liquid samples into the sample wells. The sample wells are configured to hold the liquid samples, each of the liquid samples having a second volume such that the second volume exceeds the first volume and each of the liquid sample sits within and above one of the sample wells. The slide tray and the sample tray are configured such that the slide tray can be placed onto the sample tray, at least one of the reaction sites can be positioned directly above at least one of the sample wells containing a liquid sample, and the liquid sample can be drawn onto the reaction site upon the liquid sample contacting the bottom surface of the slide.

Additional embodiments provide features that build upon the above embodiments.

### Automation Workstation

The present embodiments include a laboratory automation workstation. Laboratory automation workstations for liquid handling known in the art can be used.

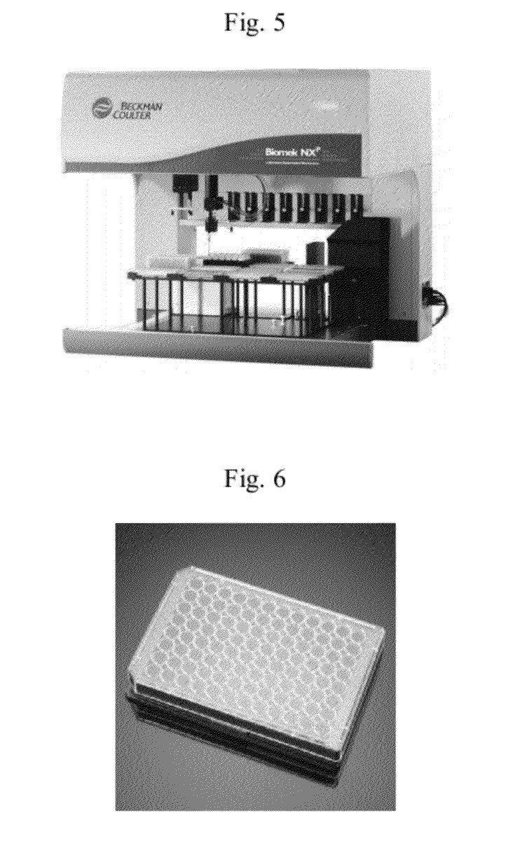

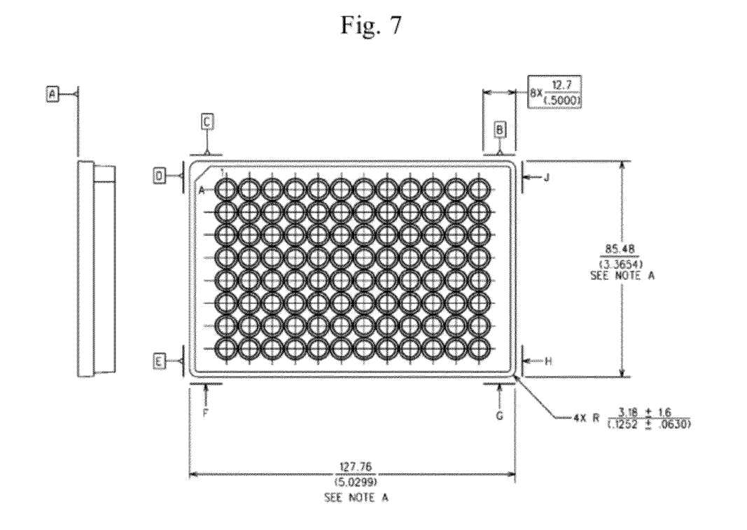

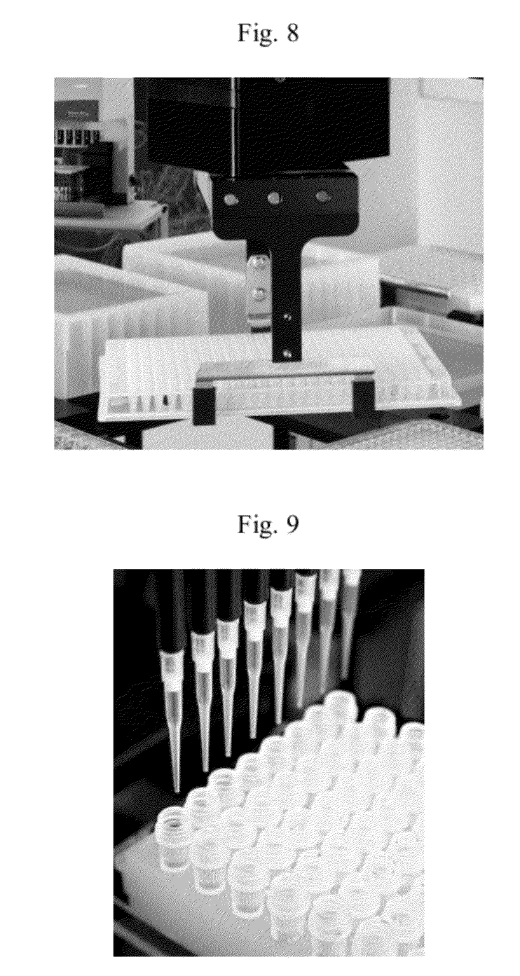

The automation workstation, for example, can manipulate, dispense liquid into, remove liquid from, shake dry, cover and store trays. The trays can have dimensions that are substantially the same as standard ANSI/SBS format titer plates, so that the automation workstation can be a titer plate automation workstation. Examples of such automation workstations include the Biomek FX^{P}, Biomek NX^{P}, Biomek 2000 and Biomek 3000, all made by Beckman Coulter, Inc. (300 N Harbor Boulevard, Fullerton, Calif. 92834-3100, U.S.A.). FIG. 5 depicts the Biomek NX^{P} Automated Workstation. FIGS. 6 and 7 depicts a standard titer plate that is commonly used with such automation workstations.

One can refer to the following titer plate related standards and dimensions and specifications cited therein: American National Standards Institute, Footprint Dimensions for Microplates, ANSI/SBS 1-2004 (Jan. 25, 2006); American National Standards Institute, Height Dimensions for Microplates, ANSI/SBS 2-2004 (Jan. 26, 2006); American National Standards Institute, Bottom Outside Flange Dimensions for Microplates, ANSI/SBS 3-2004 (Jan. 26, 2006); American National Standards Institute, Well Positions for Microplates, ANSI/SBS 4-2004 (Jan. 27, 2006).

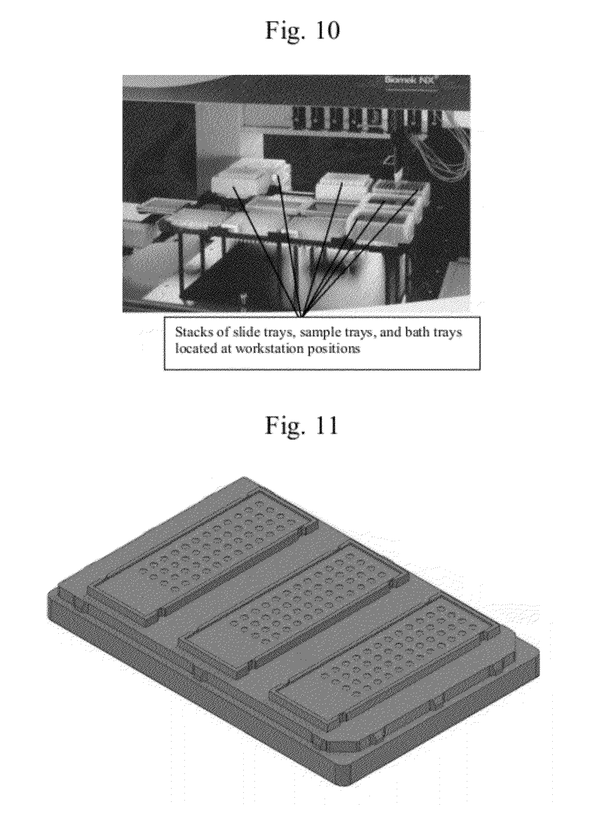

The automation workstation includes a number of workstation positions located on a deck, as shown, for example, in FIG. 10. A sample tray can be provided in, for example, a first workstation position. Also, for example, a bath tray can be provided in a second workstation position. Also, for example, a wash tray can be provided in a third workstation position. One workstation position can hold a stack of slide trays before they are transferred to the first, second or third workstation, which can be termed a slide tray input stack. One workstation position can hold a stack of slide trays after they have been transferred to the first, second or third workstation, which can be termed termed a slide tray output stack. One workstation position can hold a stack of clean sample trays, termed a sample tray input stack. One workstation position can hold a stack of used sample trays, termed a sample tray output stack. The workstation positions can include devices, such as frames, that stabilize and/or secure trays when they are placed in the workstation positions. In other embodiments, the input stacks and output stacks can be located in an area outside of the deck of the automation workstation.

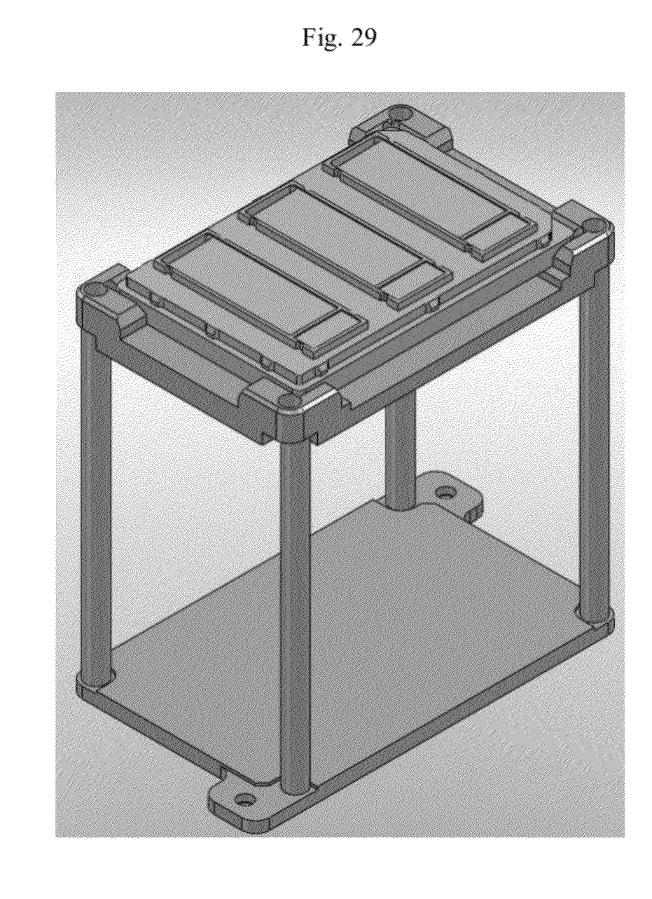

An mount may be used to hold and help process trays, as shown in FIG. 29. The automation workstation can include a gripper that can move trays from one workstation position to another. The gripper can move both vertically and horizontally. The gripper can have the ability to rotate a full 360° in a horizontal plane. FIG. 8 illustrates one such gripper, but other grippers known in the art can be used.

The automation workstation also can include a liquid dispenser. The liquid dispenser can have a single pipette to dispense liquid, or multiple pipettes as shown in FIG. 9. Other liquid dispensers known in the art can be used.

### Sample Tray

One embodiment of the sample tray is shown in FIG. 11. The sample tray has outer dimensions that allow it to be moved from one workstation position to another using the gripper. For example, the sample tray can have outer dimensions that are substantially the same as the outer dimensions of a standard ANSI/SBS format titer plate, which is depicted in FIGS. 6 and 7.

The sample tray has a sample tray surface and a recessed edge below that surface onto which a slide tray can be placed. The sample tray has one or more slide locations over which a slide is located when a slide tray is placed on the sample tray. The sample tray of FIG. 11 has 3 slide locations, but more or less slide locations are possible. Each slide location has a raised sample well surface, which is above the sample tray surface. A plurality of sample wells are located on each sample well surface. The sample wells can also be located directly on the sample tray surface rather than on the raised sample well surface (not shown). Each slide location can have an outer edge for alignment purposes.

The sample tray can be rigid or flexible. The sample tray can comprise plastics, materials having a hydrophobicity that is similar to that of plastics, or a coating of plastics and/or materials having a hydrophobicity that is similar to that of plastics. The sample tray can be surface treated if desired.

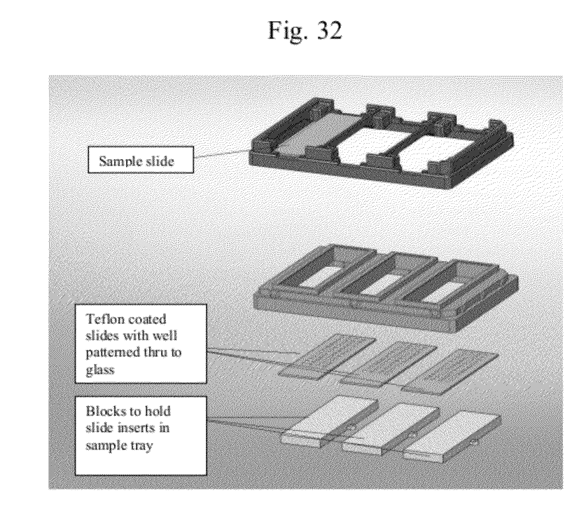

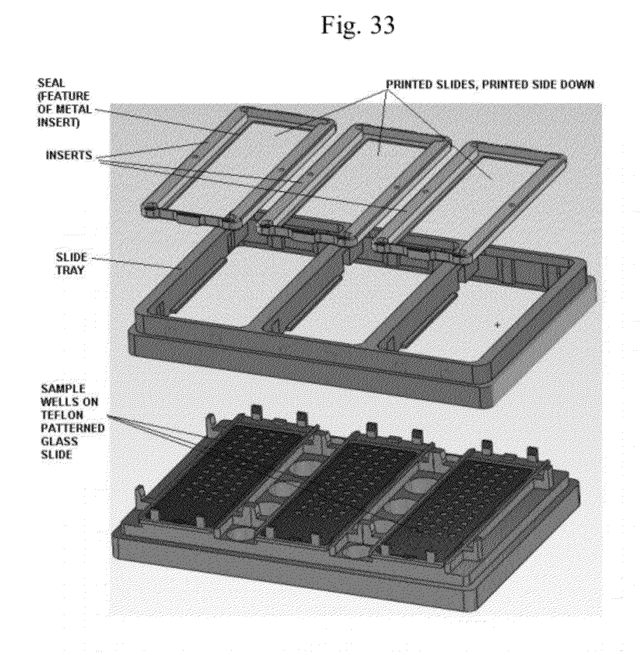

The sample tray can be made of a layer of non-hydrophobic material and a layer of hydrophobic material disposed over the layer of non-hydrophobic material, as shown for example, in FIGS. 32 and 33. The non-hydrophobic material may be, for example, glass. The hydrophobic material may be, for example, a polymer, such as polytetrafluoroethylene (e.g., Teflon). The wells are formed by circular areas where no hydrophobic material is present, such that the bare glass is exposed in the wells. The bare glass attracts the liquid while the hydrophobic material repels it. The non-hydrophobic material may be made of any suitable corrosion resistant material, such as 316 stainless steel.

In one embodiment, as depicted in FIG. 31, a machined sample slide insert can be mounted to a sample tray frame using a fastener, such as a screw, to form a sample tray. A protrusion of capture material with a hole can protrude from the side of the sample slide insert. A fastener can then be inserted through the hole and into a boss, located on the bottom side of the sample tray frame. In this embodiment, the sample tray can be made of two distinct pieces, rather than a single piece. Instead of the sample wells being located on a raised sample well surface, they are located in the sample slide insert. This allows periodic replacement of the sample slide inserts without replacing the sample tray frame or the blocks.

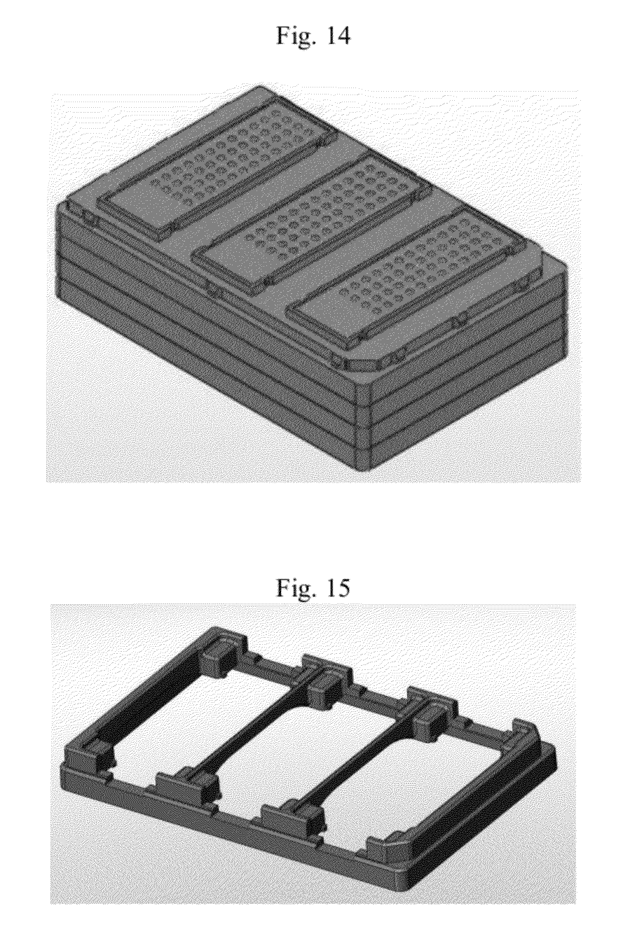

The sample tray can be cleanable and reusable or it can be disposable. The sample tray can be rectangular or square. The sample tray may be a solid piece of plastic with machined surfaces. The sample tray can also be formed using any other known plastic forming process, including injection molding. The sample tray can have a bottom surface with contours that match the contours of the recessed edge of the sample tray, making the sample trays stackable as shown in FIG. 14.

In one embodiment, as depicted in FIG. 32, a machined sample slide insert can be mounted to a sample tray frame with a block. The sample slide insert can be placed on the top surface of the block and the block can then be mounted to bottom side of the sample tray frame using a fastener, such as a screw. A protrusion of capture material with a hole can protrude from the side of the block. A fastener can then be inserted through the hole and into a boss, located on the bottom side of the sample tray frame. In this embodiment, the sample tray is made of three distinct pieces, rather than a single piece. Instead of the sample wells being located on a raised sample well surface, they are located in the sample slide insert. This allows periodic replacement of the sample slide inserts without replacing the sample tray frame or the blocks.

### Sample Wells

A sample well can also be called a recess. The number of wells can, for example, be 48, 96, or 384.

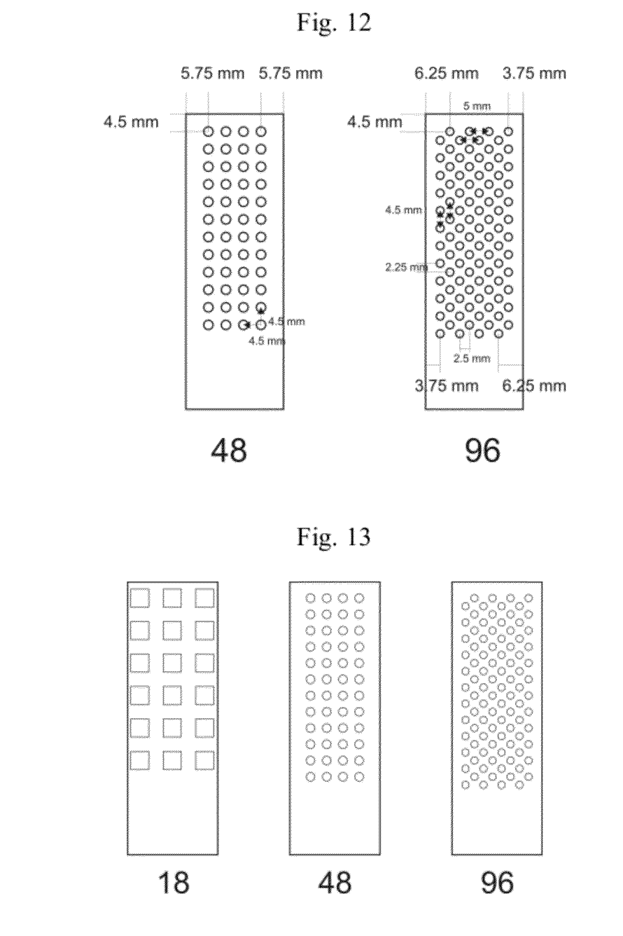

Exemplary layouts of the sample wells are shown in FIGS. 12 and 13. For example, for a 48-well sample tray, the layout of the sample wells can be a 4 by 12 array, while the distance between neighboring wells can be 4.5 mm. Layouts of sample wells known in the art for biochemical assays can be used.

The sample well can be round, rectangular, square, elliptical, or any other suitable shape. Exemplary round-shaped wells are shown in FIG. 12. Shapes of wells known in the art for biochemical assays can be used.

The sample well can be shallow. Generally, the depth of the sample well is 500 μm or less. In preferred embodiments, the depth of the sample well is 160 μm, 300 μm, or 500 μm. In one embodiment, the diameter of the well can be 2.3 mm. The size of an exemplary sample well is shown in FIG. 22. The volume of the liquid sample to be applied to the wells exceeds the volume of the well. For example, 4 μl of liquid sample is applied to each well on the 48-well sample tray, 2.5 μl of liquid sample is applied to each well on the 96-well sample tray, and 1 μl of liquid sample is applied to each well on the 384-well sample tray.

The height of the liquid sample in the sample well is larger than the depth of the well. For example, the height of the liquid sample sitting in the well can be 760 μm, while the depth of the sample well is only 160 μm, as depicted in FIG. 22. The volume of a sample well is termed a “first volume.”

Each slide tray, sample tray and bath tray can have a cavity or depression across most its bottom surface that fits over the mating top of all of the trays. The trays can share this characteristic with titer plates, which stack in this manner.

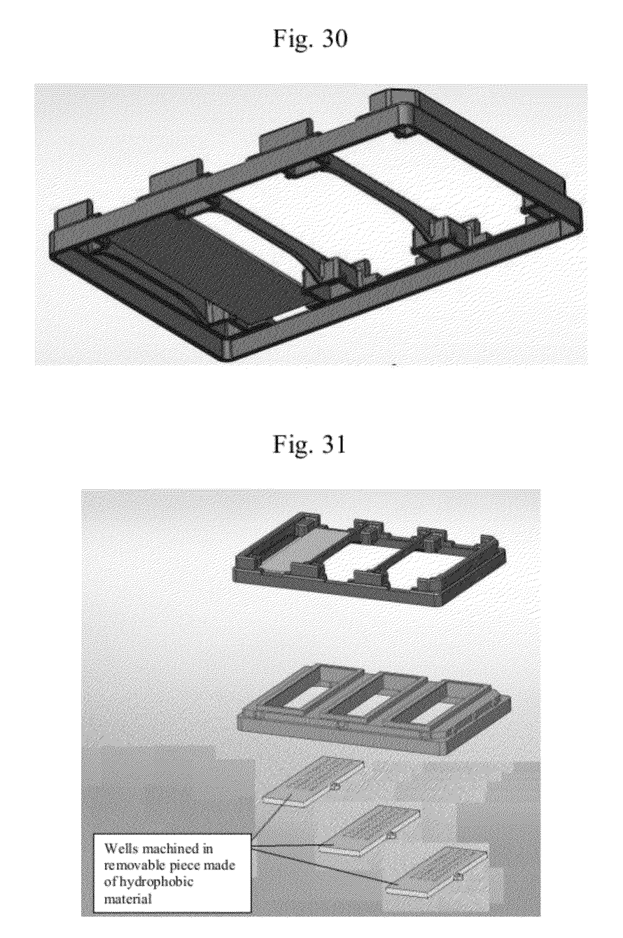

For an embodiment for the interface between the sample tray and the slide tray, see for example FIG. 30.

### Liquid Sample

Liquid samples known in the art for biochemical assays can be used. They can comprise protein or peptides, as well as nucleic acids. The liquid sample can comprise blood or urine of a human or a animal. The liquid sample can be made from tissues or cells of a human or animal. The liquid sample can be extracts of a plant or fungi. The liquid sample can comprise viruses, bacteria, or any other pathogens. The liquid sample can comprise antigens and any other analytes detectable via biochemical assays. See, for example, Alberts et al., Molecular Biology of the Cell, 5^{th} Ed., 2007 and Lodis et al., Molecular Cell Biology, 5^{th} Ed., 2007.

The volume of a liquid sample in a sample well exceeds the volume of the sample well. The volume of a liquid sample is termed a “second volume.” In preferred embodiments, the second volume is 4 μl, 2.5 μl, or 1 μl.

Several different types of liquid samples may be deposited on a single sample tray. Several different types of liquid samples may be deposited on a single slide location on a sample tray.

### Slide Tray

An exemplary slide tray is shown in FIG. 15. The slide tray has outer dimensions that enable it to be moved from one workstation position to another using the gripper. For example, the slide tray can have outer dimensions that are substantially the same as the outer dimensions of a standard ANSI/SBS format titer plate, which is depicted in FIGS. 6 and 7.

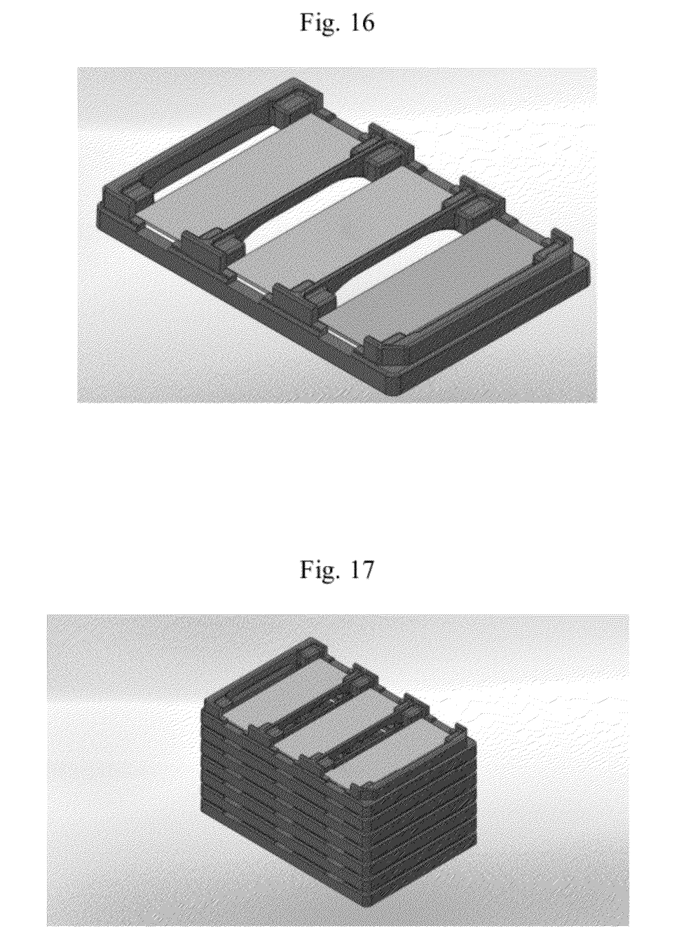

The slide tray has one or more slide locations that can each hold a single slide. A preferred embodiment, in which the slide tray holds 3 slides, is shown in FIG. 16. In a preferred embodiment, the slide can sit on small protrusions on the slide tray, as shown in FIG. 30. In another embodiment, the slide can sit on a thin lip located on at least two edges of each slide location, adjacent to the bottom surface of the slide tray. When a slide is placed into a slide location on the slide tray, the gap between the outer edges of the slide and the inner edges of the slide location can be very small such that, when the slide is placed onto the slide tray, a substantially sealed surface is created.

The slide tray has a bottom edge that allows it to be placed onto the recessed edge of the sample tray. The bottom edge of the slide tray can have contours that match the recessed edge of the sample tray. Thus, the placement of the slide tray onto the sample tray can create closed incubation chamber free of outside contamination. When the sample wells of the sample tray contain liquid samples and the slide tray is placed onto the sample tray, the bottom surface of the slides on the slide tray come into contact with the liquid samples on the sample tray. This can create a reaction volume by the reaction sites on the bottom surface of the slide. The closed chamber prevents liquid samples in the chamber from evaporating.

In one embodiment, the slide can lift off of the slide tray when the slide tray is placed on a sample tray. The slide can then sit on the edge of a slide location on the sample tray. Therefore, the distance between the bottom surface of the slide and the sample well surface of the sample tray will depend on the height of the edge on which the slide sits. When the slide is lifted off the slide tray onto the edge, a closed incubation chamber is created between the bottom surface of the slide and the sample well surface of the sample tray. In other embodiments, this same configuration is used when the slide tray is placed on a bath tray or wash tray.

The slide tray can be rigid or flexible. The slide tray can comprise plastics, materials having a hydrophobicity that is similar to that of plastics, or a coating of plastics and/or materials having a hydrophobicity that is similar to that of plastics. The slide tray can be surface treated if desired.

The slide tray can be cleanable and reusable or it can be disposable. The slide tray can be called a chip or a substrate platform. The slide tray can be rectangular or square. In a preferred embodiment, the slide tray is a solid piece of plastic with machined surfaces. The slide tray can also be formed using any other known plastic forming process, including injection molding. The slide tray can have a bottom surface with contours that match the contours of an outer edge of the slide tray, making the slide trays stackable as shown in FIG. 17.

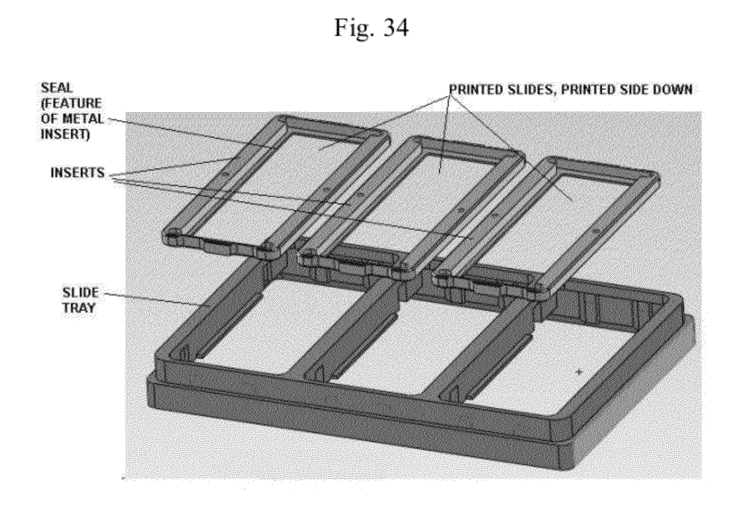

Slides may be placed in inserts prior to being loaded into the slide tray, as shown in FIG. 34. The inserts may be made of metal. The inserts may comprise a seal portion. This allows the slide/insert to independently rest on the surface of the sample tray, bath tray, or wash tray, providing a good seal between the printed slide and the well slide.

### Slide

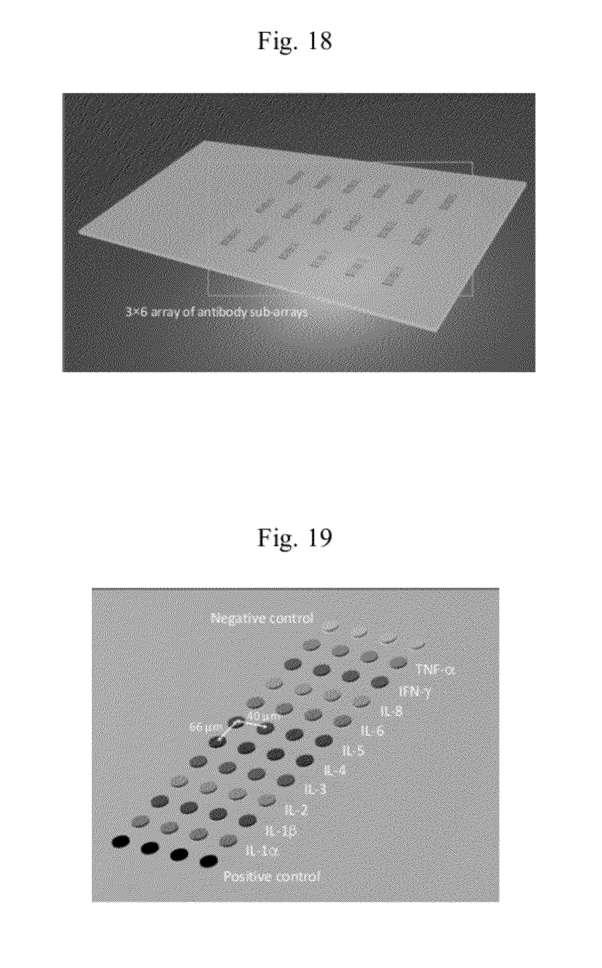

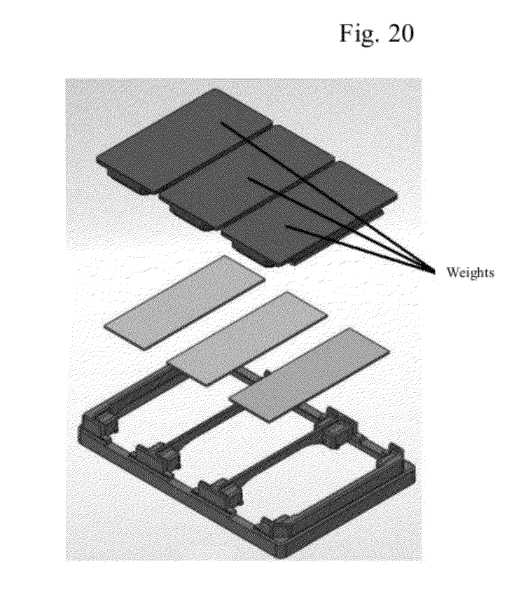

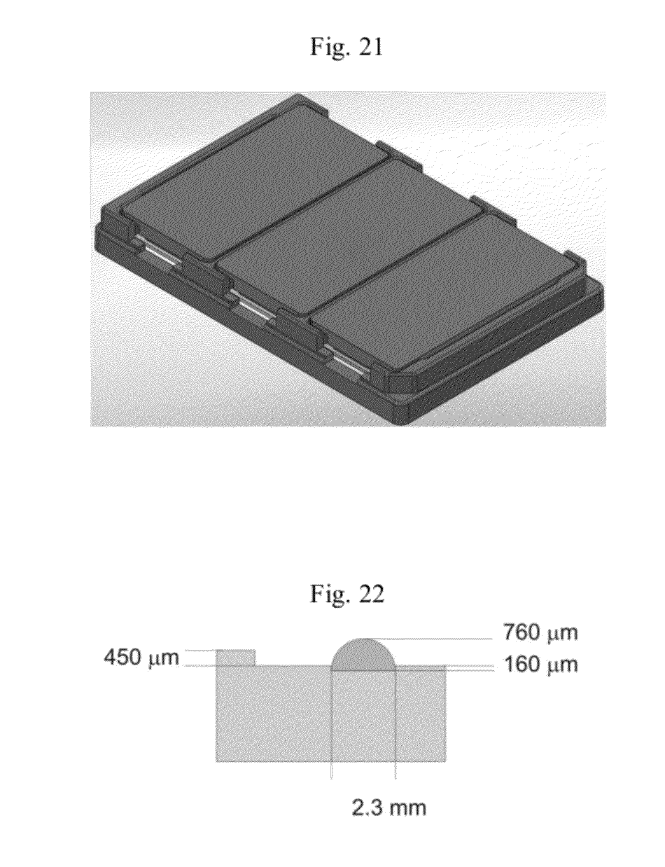

Slides known in the art for biochemical assays can be used. The slide can be rigid or flexible. It can be flat. The slide can be rectangular or square. Exemplary slides are shown in FIGS. 18 and 19.

The slide can comprise glass, materials having a similar hydrophobicity as glass, or a coating of glass and/or materials having a similar hydrophobicity as glass. The slide can be surface treated if desired.

The slide can also be called a microarray, as a array of reaction sites is printed on the bottom surface of the slide. One preferred embodiment of the slide is a solid piece of epoxy glass printed with an array of antibodies via a Dip Pen Nanolithography (DPN) process.

When the slide is placed on the sample tray, the bottom surface of the slide faces the sample tray surface and the raised sample well surface. The distance between the bottom surface of the slide and the sample well surface is such that the top of the liquid sample sitting in a sample well will contact the bottom surface of the slide. In other words, the distance from the bottom of the sample well to the top of the liquid sample when the liquid sample is in the sample well is greater than the distance between the bottom of the sample well and the bottom surface of the slide when the slide tray is placed on the sample tray.

The bottom surface of the slide is preferably hydrophilic while both the top surface of the sample tray and the surface of the sample wells are preferably hydrophobic.

### Reaction Sites or Sub-Arrays

The slide can comprise reaction sites or sub-arrays. Reaction sites and sub-arrays known in the art for biochemical assays can be used. They can comprise antibodies generated from immune responses of a human or animal. The reaction sites or sub-array can bind specifically to one or more antigens or any other analytes detectable via biochemical assays. See, for example, Alberts et al., Molecular Biology of the Cell, 5^{th} Ed., 2007 and Lodish et al., Molecular Cell Biology, 5^{th} Ed., 2007.

In a preferred embodiment, the reaction sites or sub-arrays comprise antibodies printed on a glass slide via a DPN process. The DPN method is described in U.S. Pat. Nos. 6,635,311; 6,827,979; and 7,744,963 (Mirkin et al.).

The layout of the reaction sites on the slide preferably mirrors the layouts of the sample wells on the sample tray. Consequently, in a preferred embodiment, when the slide tray is placed on the sample tray, each reaction site printed on the bottom of the slide will be positioned directly above each corresponding sample well on the sample tray.

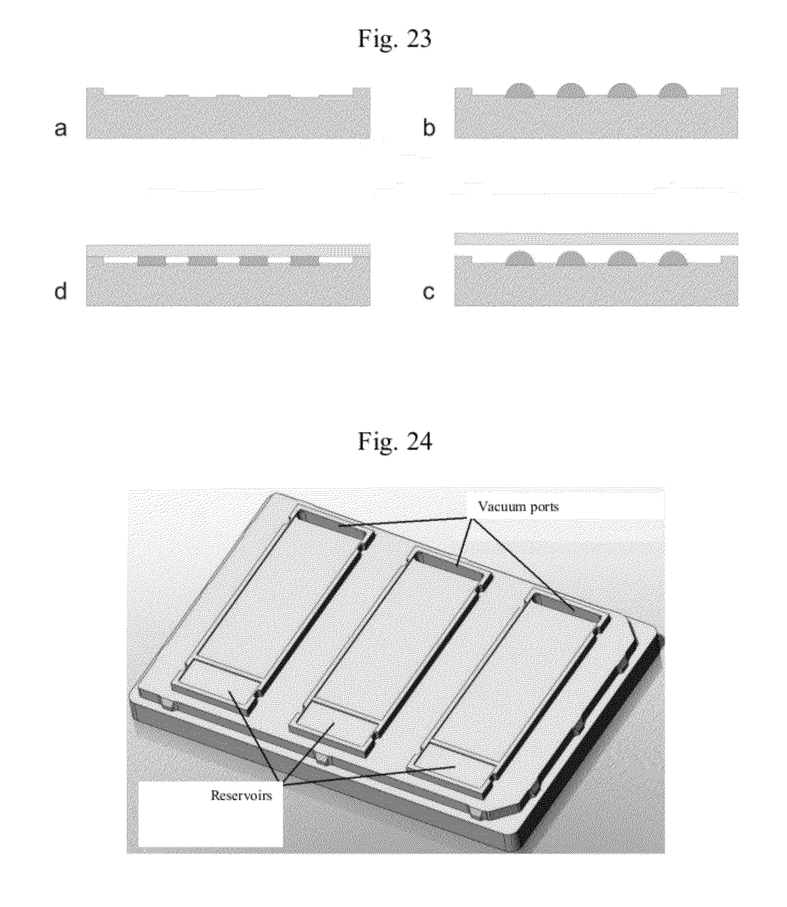

When the slide is placed on the sample tray, liquid samples sitting in the sample wells will make contact with the bottom surface of the slide. Because of the hydrophobicity of the bottom surface of the slide exceeds the hydrophobicity of both the sample well surface of the sample tray and the surface of the sample well, the liquid sample is drawn upwards upon contacting the bottom surface of the slide. This is shown in FIGS. 23(a) to 23(d).

In a preferred embodiment, when a slide tray is placed on the sample tray, each reaction site printed on the bottom of the slide is positioned directly above each sample well on the sample tray. Thus, upon contacting the bottom surface of the slide, the liquid sample is drawn upwards to form a reaction volume on the reaction site directly above it.

### Bath Tray

A bath tray may be used for bulk exposure of slides to a single liquid. An exemplary bath tray is shown in FIG. 24. The bath tray has outer dimensions that enable it to be moved from one workstation position to another using the gripper. For example, the bath tray can have outer dimensions that are substantially the same as the outer dimensions of a standard ANSI/SBS format titer plate, which is depicted in FIGS. 6 and 7.

The bath tray has a bath tray surface and a recessed edge below that surface onto which a slide tray can be placed. The bath tray has one or more slide locations over which a slide is located when a slide tray is placed on the sample tray. The bath tray of FIG. 24 has 3 slide locations, but more or less slide locations are possible. Each slide location has a raised bath well surface, which is above the bath tray surface. A bath well is located on each bath well surface. The bath wells can also be located directly on the bath tray surface rather than on the raised bath well surface (not shown).

Each slide location may have a reservoir into which the liquid dispenser dispenses a bath liquid, as shown in FIG. 24. The reservoirs feed the bath wells.

The bath tray does not necessarily have a unique well for each reaction site. Instead, all of the reaction sites may exposed to the same bath liquid. When such a exposure is required, the automation workstation can move a slide tray onto a bath tray using the gripper. The liquid dispenser can deposit the bath liquid into the reservoir, which feeds the bath wells. The bath liquid thereby contacts the reaction sites on the slides.

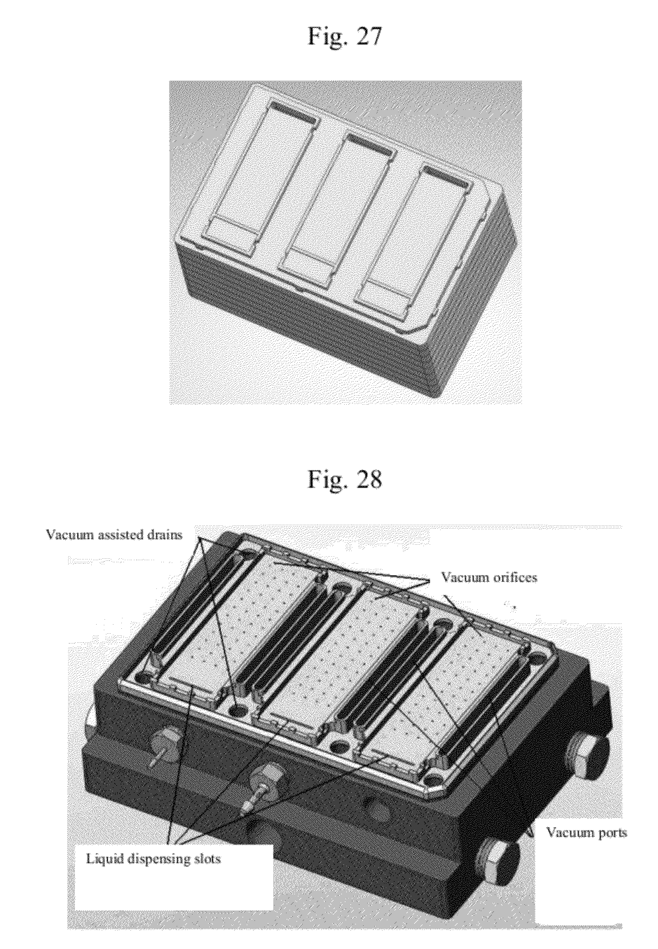

Each slide location may comprise a vacuum port. A vacuum device can create a vacuum in the chamber created between the bath tray and the slide tray. Vacuums known in the art can be used. The vacuum can assist capillary action to allow the bath liquid to contact the reaction sites. The vacuum can also be used to help dry the slide. Other embodiments do not include a reservoir or vacuum port (FIG. 27). Other embodiments can include a slotted bath well surface (FIG. 26).

Each slide location can have an outer edge for alignment purposes.

The bath tray can be rigid or flexible. The bath tray can comprise plastics, materials having a hydrophobicity that is similar to that of plastics, or a coating of plastics and/or materials having a hydrophobicity that is similar to that of plastics. The bath tray can be surface treated if desired.

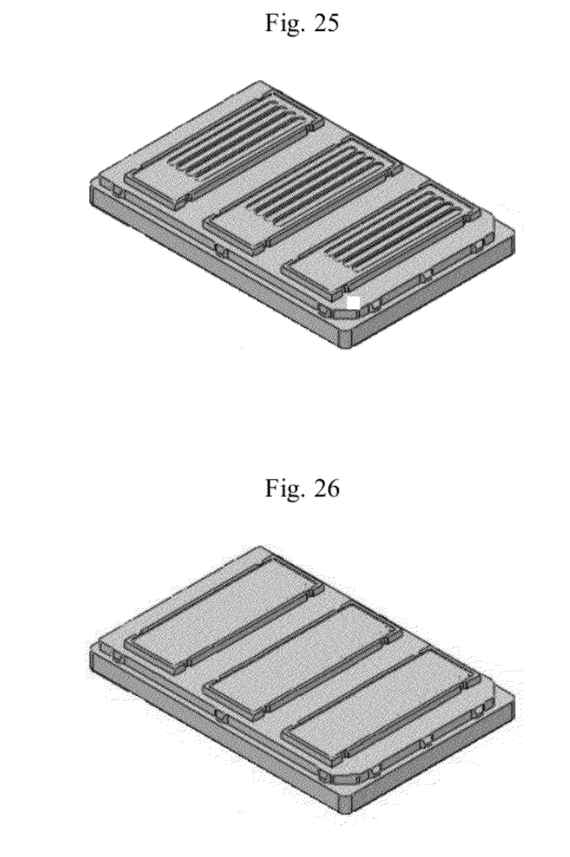

The bath tray can be cleanable and reusable or it can be disposable. The bath tray can be rectangular or square. In a preferred embodiment, the bath tray is a solid piece of plastic with a machined top surface. The bath tray can also be formed using any other known plastic forming process, including injection molding. The bath tray can have a bottom surface with contours that match the contours of the recessed edge of the bath tray, making the bath trays stackable as shown in FIG. 25. The interfaces between titer plates, which are known in the art and often subject to standardization, are known in the art and can be adapted for embodiments described herein.

In some embodiments, a sample tray may be used for bulk exposure of slides to a single liquid, rather than a bath tray. In this case, each of the wells in the sample tray may simply be filled with the same liquid.

### Wash Tray

An exemplary wash tray on a wash station is shown in FIG. 28. In a preferred embodiment, the wash tray is permanently attached to the wash station. The wash tray can, however, be movable and have outer dimensions that enable it to be moved from one workstation position to another using the gripper (not shown). For example, the wash tray can have outer dimensions that are substantially the same as the outer dimensions of a standard ANSI/SBS format titer plate, which is depicted in FIGS. 6 and 7.

The wash tray of FIG. 28 has a wash tray surface. The wash tray has one or more slide locations over which a slide is located when a slide tray is placed on the sample tray. The wash tray of FIG. 28 has 3 slide locations, but more or less slide locations are possible. Each slide location has a raised wash well surface, which is above the wash tray surface. A wash well is located on each wash well surface. The wash wells can also be located directly on the bath tray surface rather than on the raised wash well surface (not shown).

In a preferred embodiment, depicted in FIG. 28, each slide location has a slots on the wash well surface through which wash buffer is deposited.

Unlike the sample tray, the wash tray does not have a unique well for each reaction site. All reaction sites are exposed to the same wash buffer. When such a exposure is required, the automation workstation can move a slide tray onto a wash tray using the gripper. In a preferred embodiment, a second liquid dispenser deposits wash buffer through the slots. The wash buffer thereby contacts the reaction sites on the slides. In other embodiments, the wash buffer can be deposited using the same liquid dispenser that deposits the liquid samples and the bath liquid (not shown).

In a preferred embodiment, the wash tray includes a series of small orifices connected to a vacuum device that can vacuum liquid out of the wash well.

The wash tray can include a series of vacuum assisted drains, shown in FIG. 28, to allow wash buffer to drain.

The wash tray can include a series of vacuum ports, shown in FIG. 28, that can pull slide tray weights an a slide tray downward, sealing the top of the slide to keep it dry.

Each slide location can have an outer edge for alignment purposes.

The wash tray can be rigid or flexible. The wash tray can comprise plastics, materials having a hydrophobicity that is similar to that of plastics, or a coating of plastics and/or materials having a hydrophobicity that is similar to that of plastics. The wash tray can be surface treated if desired. The wash tray can be cleanable and reusable or it can be disposable. The wash tray can be rectangular or square. In a preferred embodiment, the wash tray is a solid piece of plastic with a machined top surface. The wash tray can also be formed using any other known plastic forming process, including injection molding. The wash tray can have a bottom surface with contours that match the contours of the recessed edge of the wash tray, making the wash trays stackable.

### Washing and Drying Stations

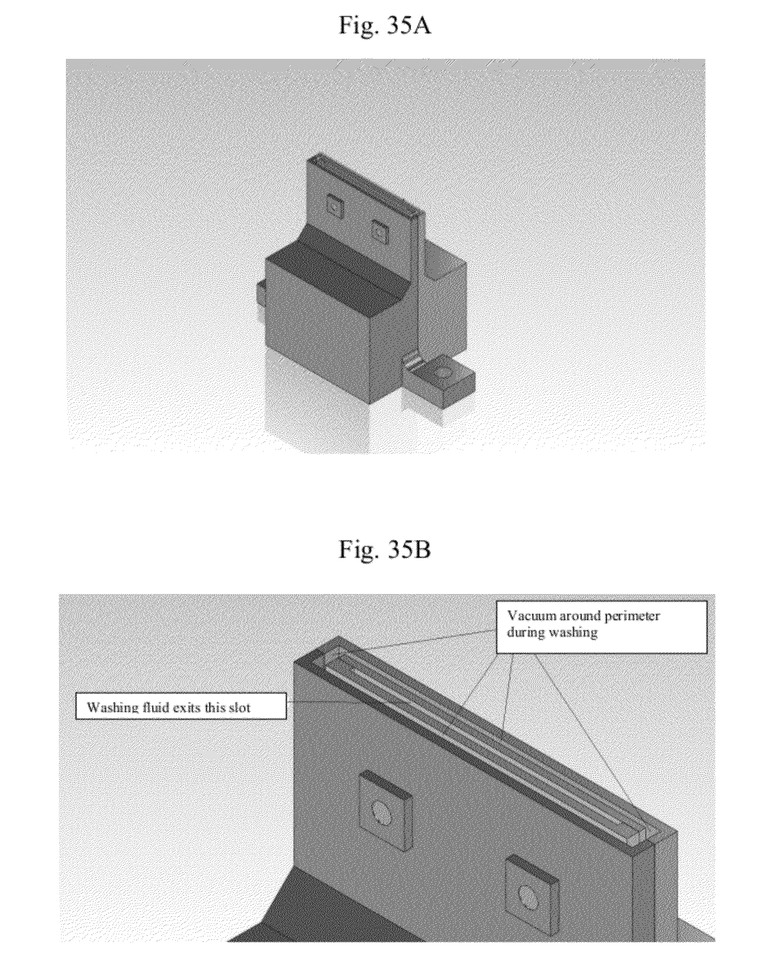

As an alternative to the wash tray, washing of slides using buffer solution, de-ionized water or other fluids may be performed in a washing station via a nozzle that moves back and forth across the printed side of the sample slide. An embodiment of the nozzle is shown in FIGS. 35A and 35B. To reduce the amount of fluid that spills out over the edges of the slides, a light vacuum is pulled all around the periphery of the nozzle.

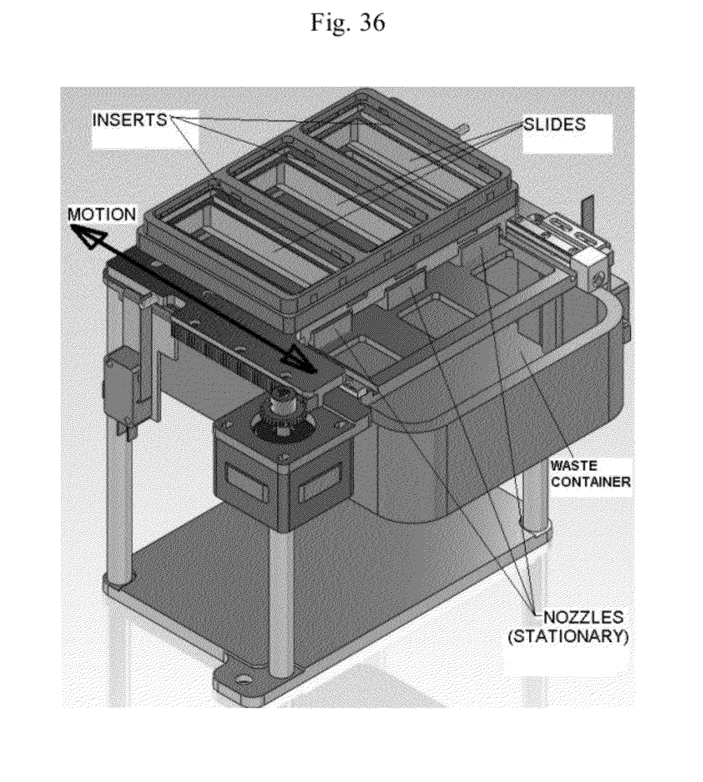

There can be, for example, three slides and three nozzles in the washing station, as shown in FIG. 36. To wash the slide, the slide tray is set onto a washing station that contains the nozzles. Once the slide tray, with its printed slides, is in place on the washing station, the automation workstation starts the flow of fluid through the three nozzles. The automation workstation then moves the nozzles back and forth across the entire length of printed area of the slide a number of times to wash the slide.

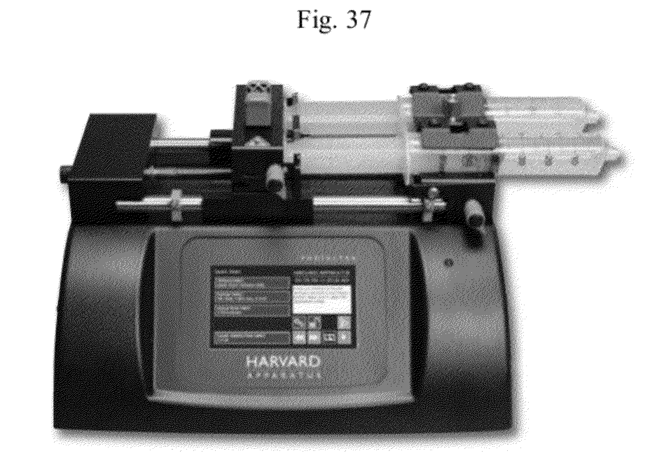

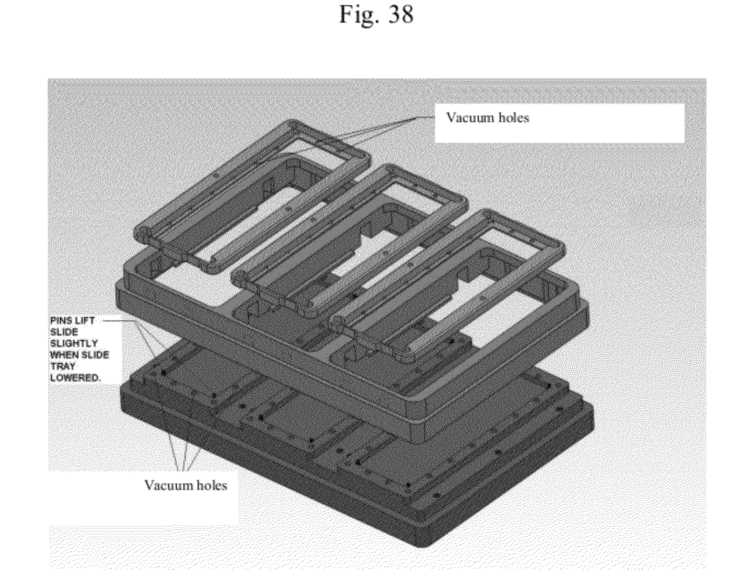

Controlled flow of washing fluid into the nozzles is important. Syringe pumps, peristaltic pumps, and air pressure pumps may be used, with syringe pumps being preferred due to their accurate control of fluid flow rate and amount. An example of such a syringe pump is shown in FIG. 37.

At the end of processing, the slides in a slide tray may be dried using a drying station. The drying station may be identical to the washing station, except that the station's nozzles are optimized for drying with a higher vacuum level.

Alternatively, a “peripheral vacuum” drying station with no moving parts may be used, such as the one shown in FIG. 38. The drying station may have a base with a plurality of vacuum holes that feed vacuum to a plurality of holes in an insert around a slide in the insert.

### Literature

Additional applications and teachings are described in the following references:

### Non-Patent Literatures:

1. Huang et al., “High-throughput genomic and proteomic analysis using microarray technology,” Clinical Biochmistry, 47(10):1912-1916 (2001).

2. Dunn & Feygin, “Challenges and solutions to ultra-high-throughput screening assay miniaturization: submicroliter fluid handling, DDT, 5(12):S84-S91 (2000).

3. Templin et al., “Protein microarray technology,” Trends in Biotechnology, 20(4):160-166 (2002).

4. Heller, “DNA microarray technology,” Annu. Rev. Biomed. Eng., 4:129-153 (2002).

5. Ochsner et al., “Micro-well arrays for 3D shape control and high resolution analysis of single cells,” Lab Chip, 7:1074-1077 (2007).

6. Khademhosseini et al., “Co-culture of human embryonic stem cells with murine embroni fibroblasts on microwell-patterned substrates,” Biomaterials, 27:5968-5977 (2006).

### Patent or Published Patent Applications:

1. U.S. Pat. No. 7,736,594, “Reaction surface array diagnostic apparatus.”

2. U.S. Pat. No. 7,666,362, “Micro-plate and lid for robotic handling.”

3. U.S. Pat. No. 7,166,257, “Multiwell test apparatus.”

4. U.S. Pat. No. 7,128,878, “Multiwell plate.”

5. U.S. Pat. No. 6,939,709, “Multi-well device.”

6. U.S. Pat. No. 6,720,143, “Genetic assay system.”

7. U.S. Pat. No. 6,699,665, “Multiple array system for integrating bioarrays.”

8. U.S. Pat. No. 6,436,050, “Multi-well platforms, caddies, lids and combinations thereof”

9. U.S. Pat. No. 6,303,387, “Method of transferring a liquid drop from a multiwell plate and/or chemical assay.”

10. U.S. Pat. No. 6,037,168, “Microbiological assembly comprising resealable closure means.”

11. U.S. Pat. No. 5,972,694, “Multi-well plate.”

12. U.S. Pat. No. 6,703,247, “Apparatus and Methods for Efficient Processing of Biological Samples on Slides.”

13. U.S. Pat. No. 3,736,042, “Microscope slide assembly.”

14. U.S. Pat. No. 5,654,200, “Automated Slide Processing Apparatus with Fluid Injector.”

15. U.S. Pat. No. 5,948,359, “Automated Staining Apparatus.”

16. U.S. Pat. No. 5,473,706, “Method and Apparatus for Automated Assay of Biological Specimens.”

17. U.S. Patent Publication No. 2008/0038836, “Automated High Volume Slide Staining System.”

18. U.S. Patent Publication No. 2005/0186114, “Automated High Volume Slide Processing System.”

19. U.S. Patent Publication No. 2004/0049351, “Immunosorbent Assay in Microarray Format.”

What is claimed is:

1. A method comprising:
providing an automation workstation comprising a gripper, a liquid dispenser, and a plurality of workstation positions,
providing a sample tray in a first workstation position, wherein the sample tray comprises a plurality of sample wells having a first volume,
providing a slide tray comprising at least one slide, wherein the slide comprises a plurality of reaction sites on a bottom surface of the slide,
depositing a liquid sample having a second volume into at least one of the sample wells using the liquid dispenser such that the second volume exceeds the first volume and the liquid sample sits within and above the at least one sample well,
moving the slide tray to the first workstation position using the gripper, and
placing the slide tray onto the sample tray using the gripper such that at least one of the reaction sites is positioned above the at least one sample well containing the liquid sample and the liquid sample is drawn onto the reaction site upon the liquid sample contacting the bottom surface of the slide.

2. The method of claim 1, further comprising:
providing a second tray in a second workstation position, wherein the second tray is a second sample tray or a bath tray,
depositing a second liquid sample or a bath liquid into at least one well of the second tray,
moving the slide tray to the second workstation position using the gripper,
placing the slide tray onto the second tray using the gripper.

3. The method of claim 1, further comprising:
providing a washing station in a second workstation position, wherein the washing station comprises at least one nozzle
moving the slide tray to the second workstation position using the gripper,
placing the slide tray onto the washing station using the gripper, and
moving the at least one nozzle across the slide while spraying washing fluid from the nozzle onto the slide.

4. The method of claim 3, wherein the at least one nozzle comprises a first slot configured to supply the washing fluid and a second slot configured to supply a vacuum, and wherein the method further comprises creating a vacuum using the nozzle to vacuum at least a portion of the washing fluid.

5. The method of claim 3, wherein a flow of the washing fluid is controlled using a syringe pump.

6. The method of claim 3, wherein the washing station is permanently attached at the second workstation position.

7. The method of claim 3, further comprising:
providing a drying station in a third workstation position, wherein the drying station comprises at least one vacuum nozzle configured to provide a vacuum,
moving the slide tray to the third workstation position using the gripper,
placing the slide tray onto the drying station using the gripper, and
moving the at least one vacuum nozzle across the slide while providing the vacuum from the vacuum nozzle to the slide.

8. The method of claim 1, further comprising the step of placing a weight on the slides in the slide tray.

9. The method of claim 1, further comprising:
providing a wash tray in a second workstation position, wherein the wash tray comprises a wash well,
moving the slide tray to the second workstation position using the gripper,
placing the slide tray onto the wash tray using the gripper, and
depositing a wash buffer into the wash well such that the wash buffer contacts the plurality of reaction sites.

10. The method of claim 1, wherein the slide trays are stackable.

11. The method of claim 10, wherein the slide tray is moved to the first workstation position from a slide tray input stack using the gripper.

12. The method of claim 10, further comprising the step of moving the slide tray to a slide tray output stack using the gripper.

13. The method of claim 1, wherein the sample trays are stackable.

14. The method of claim 13, further comprising the step of moving the sample tray to the first workstation position from a sample tray input stack using the gripper.

15. The method of claim 13, further comprising the step of moving the sample tray to a sample tray output stack using the gripper.

16. The method of claim 1, wherein the slide tray comprises at least one additional slide.

17. The method of claim 1, wherein the sample tray is made of plastic.

18. The method of claim 17, wherein the sample tray is made of a solid piece of plastic.

19. The method of claim 18, wherein the sample tray is of rectangular shape.

20. The method of claim 1, wherein the number of sample wells is selected from the group consisting of 48, 96, and 384.

21. The method of claim 1, wherein the distance between neighboring sample wells is about 4.5 mm.

22. The method of claim 1, wherein the sample wells are round.

23. The method of claim 1, wherein the depth of the sample wells is less that 500 μm.

24. The method of claim 1, wherein the depth of the sample wells is less than 300 μm.

25. The method of claim 1, wherein the depth of the sample wells is less than 160 μm.

26. The method of claim 1, wherein the liquid dispenser comprises a pipette.

27. The method of claim 1, wherein the first volume is less than 2.5 μl.

28. The method of claim 1, wherein the first volume is less than 1 μl.

29. The method of claim 1, wherein the second volume is selected from a group consisting of 4 μl, 2.5 μl, or 1 μl.

30. The method of claim 1, wherein the distance from the bottom of the sample well to the top of the liquid sample when the liquid sample is in the well is greater than the distance between the bottom of the sample well and the bottom surface of the slide when the slide tray is placed on the sample tray.

31. The method of claim 1, wherein the liquid sample comprises analytes capable of being captured by the reaction sites and the reaction sites comprise capture molecules capable of capturing analytes.

32. The method of claim 1, wherein the liquid sample comprises antigens and wherein the reaction site comprises antibodies.

33. The method of claim 1, wherein the automation workstation is a titer plate laboratory workstation.

34. The method of claim 1, wherein the slide comprises a glass material.

35. The method of claim 34, wherein the slide comprises a solid piece of epoxy glass.

36. The method of claim 35, wherein the reaction sites comprise antibodies.

37. The method of claim 1, wherein the reaction sites are printed onto the slide via a Dip Pen Nanolithography process.

38. The method of claim 1, wherein the positions of the reaction sites match the positions of the sample wells.

39. The method of claim 1, wherein the bottom surface of the slide comprises a hydrophilic material.

40. The method of claim 1, wherein the liquid sample creates a reaction volume over one of the reaction sites upon contacting the bottom surface of the slide.

41. The method of claim 1, further comprising the step of securing the slide to the slide tray.

42. The method of claim 41, wherein the slide is secured to the slide tray using a screw.

43. The method of claim 1, wherein the sample slide comprises a glass layer and a polymer layer disposed on the glass layer, wherein the plurality of sample wells are formed by a plurality of circular areas at which the glass layer is exposed through the polymer layer.

44. The method of claim 43, wherein the polymer layer is made of polytetrafluoroethylene.

45. The method of claim 1, wherein the slide tray further comprises at least one insert configured to hold the at least one slide, wherein the at least one insert is removable from the slide tray and the at least one slide is removable from the insert.

46. An article comprising:
an automation workstation comprising a gripper, a liquid dispenser, and a plurality of workstation positions,
a sample tray configured to be placed in a first workstation position, wherein the sample tray comprises a plurality of sample wells having a first volume,
a slide tray comprising a slide, wherein the slide comprises a plurality of reaction sites on a bottom surface of the slide,
wherein the liquid dispenser is configured to deposit a plurality of liquid samples into the sample wells,
wherein the sample wells are configured to hold the liquid samples, each of the liquid samples having a second volume such that the second volume exceeds the first volume and each of the liquid sample sits within and above one of the sample wells,
wherein the slide tray is configured to be movable to the first workstation position using the gripper, and
wherein the slide tray and the sample tray are configured such that the slide tray can be placed onto the sample tray using the gripper, at least one of the reaction sites can be positioned above at least one of the sample wells containing a liquid sample, and the liquid sample can be drawn onto the reaction site upon the liquid sample contacting the bottom surface of the slide.

47. The article of claim 46, further comprising:
a second tray configured to be placed in a second workstation position, wherein the second tray is a second sample tray or a bath tray,
wherein the liquid dispenser is configured to deposit a second liquid sample or a bath liquid into at least one well of the second tray,
wherein the slide tray is configured to be movable to the second workstation position and placed on the second tray using the gripper.

48. The article of claim 46, further comprising:
a washing station configured to be placed in a second workstation position, wherein the washing station comprises at least one nozzle,
wherein the slide tray and the washing station are configured such that the slide tray can be moved to the second workstation position and placed on the washing station using the gripper, and
wherein the nozzle is configured to move across the slide while spraying washing fluid from the nozzle onto the slide.

49. The article of claim 48, wherein the at least one nozzle comprises a first slot configured to supply the washing fluid and a second slot configured to supply a vacuum, and wherein the nozzle is configured to create a vacuum to vacuum at least a portion of the washing fluid.

50. The article of claim 48, further comprising a syringe pump configured to control a flow of washing fluid from the nozzle.

51. The article of claim 48, wherein the wash tray is permanently attached at the second workstation position.

52. The article of claim 48, further comprising:
a drying station configured to be placed in a third workstation position, wherein the drying station comprises at least one vacuum nozzle configured to provide a vacuum,
wherein the slide tray and the washing station are configured such that the slide tray can be moved to the third workstation position and placed on the drying station using the gripper, and
wherein the vacuum nozzle is configured to move across the slide while providing a vacuum from the vacuum nozzle to the slide.

53. The article of claim 46, further comprising a weight configured to be placed on the slides in the slide tray.

54. The article of claim 46, further comprising:
a wash tray configured to be placed in a second workstation position, wherein the wash tray comprises a wash well,
wherein the wash tray is configured to be movable to the second workstation position using the gripper, and
wherein the slide tray and the wash tray are configured such that the slide tray can be placed on the wash tray using the gripper and wash buffer can be deposited into the wash well such that the wash buffer contacts the plurality of reaction sites.

55. The article of claim 46, wherein the slide trays are stackable.

56. The article of claim 55, wherein the slide tray is configured to be movable to the first workstation position from a slide tray input stack using the gripper.

57. The article of claim 55, wherein the slide tray is configured to be movable to a slide tray output stack using the gripper.

58. The article of claim 46, wherein the sample trays are stackable.

59. The article of claim 58, wherein the sample tray is configured to be movable to the first workstation position from a sample tray input stack using the gripper.

60. The article of claim 58, wherein the sample tray is configured to be movable to a sample tray output stack using the gripper.

61. The article of claim 46, wherein the slide tray comprises at least one additional slide.

62. The article of claim 46, wherein the sample tray is made of plastic.

63. The article of claim 62, wherein the sample tray is made of a solid piece of plastic.

64. The article of claim 62, wherein the sample tray is of rectangular shape.

65. The article of claim 46, wherein the number of sample wells is selected from the group consisting of 48, 96, and 384.

66. The article of claim 46, wherein the distance between neighboring sample wells is about 4.5 mm.

67. The article of claim 46, wherein the sample wells are round.

68. The article of claim 46, wherein the depth of the sample wells is less that 500 μm.

69. The article of claim 46, wherein the depth of the sample wells is less than 300 μm.

70. The article of claim 46, wherein the depth of the sample wells is less than 160 μm.

71. The article of claim 46, wherein the liquid dispenser comprises a pipette.

72. The article of claim 46, wherein the first volume is less than 2.5 μl.

73. The article of claim 46, wherein the first volume is less than 1 μl.

74. The article of claim 46, wherein the second volume is selected from a group consisting of 4 μl, 2.5 μl, or 1 μl.

75. The article of claim 46, wherein the distance from the bottom of the sample well to the top of the liquid sample when the liquid sample is in the well is greater than the distance between the bottom of the sample well and the bottom surface of the slide when the slide tray is placed on the sample tray.

76. The article of claim 46, wherein the liquid sample comprises analytes capable of being captured by the reaction sites and the reaction sites comprise capture molecules capable of capturing analytes.

77. The article of claim 46, wherein the liquid sample comprises antigens and wherein the reaction site comprises antibodies.

78. The article of claim 46, wherein the automation workstation is a titer plate laboratory workstation.

79. The article of claim 46, wherein the slide comprises a glass material.

80. The article of claim 79, wherein the slide comprises a solid piece of epoxy glass.

81. The article of claim 80, wherein the reaction sites comprise an array of antibodies.

82. The article d of claim 46, wherein the reaction sites are printed onto the slide via a Dip Pen Nanolithography process.

83. The article of claim 46, wherein the positions of the reaction sites match the positions of the sample wells.

84. The article of claim 46, wherein the bottom surface of the slide comprises a hydrophilic material.

85. The article of claim 46, wherein the liquid sample creates a reaction volume over one of the reaction sites upon contacting the bottom surface of the slide.

86. The article of claim 46, further comprising a fastener configured to secure the slide to the slide tray.

87. The article of claim 86, wherein the fastener is a screw.

88. The article of claim 46, wherein the sample slide comprises a glass layer and a polymer layer disposed on the glass layer, wherein the plurality of sample wells are formed by a plurality of circular areas at which the glass layer is exposed through the polymer layer.

89. The article of claim 88, wherein the polymer layer is made of polytetrafluoroethylene.

90. The article of claim 46, wherein the slide tray further comprises at least one insert configured to hold the at least one slide, wherein the at least one insert is removable from the slide tray and the at least one slide is removable from the insert.

91. An article comprising:
a sample tray comprising a plurality of sample wells having a first volume,
a slide tray comprising a slide, wherein the slide comprises a plurality of reaction sites on a bottom surface of the slide,
a liquid dispenser configured to dispense a plurality of liquid samples into the sample wells,
wherein the sample wells are configured to hold the liquid samples, each of the liquid samples having a second volume such that the second volume exceeds the first volume and each of the liquid sample sits within and above one of the sample wells,
wherein the slide tray and the sample tray are configured such that the slide tray can be placed onto the sample tray, at least one of the reaction sites can be positioned directly above at least one of the sample wells containing a liquid sample, and the liquid sample can be drawn onto the reaction site upon the liquid sample contacting the bottom surface of the slide.

92. The article of claim 91, wherein:
the sample tray has outside dimensions that are substantially similar to the outside dimensions of a standard titer plate, and
the slide tray has outside dimensions that are substantially similar to the outside dimensions of a standard titer plate.

93. The method of claim 1, further comprising:
providing a bath tray in a second workstation position, wherein the bath tray comprises a bath well,
moving the slide tray to the second workstation position using the gripper,
placing the slide tray onto the bath tray using the gripper, and
depositing a bath liquid into the bath well using the liquid dispenser such that the bath liquid contacts the plurality of reaction sites.

94. The method of claim 93, further comprising:
providing a wash tray in a third workstation position, wherein the wash tray comprises a wash well,
moving the slide tray to the third workstation position using the gripper,
placing the slide tray onto the wash tray using the gripper, and
depositing a wash buffer into the wash well such that the wash buffer contacts the plurality of reaction sites.

95. The method of claim 94, wherein the wash buffer is deposited using the liquid dispenser of claim 1.

96. The method of claim 94, wherein the wash buffer is deposited using a second liquid dispenser.

97. The method of claim 94, wherein the wash tray is permanently attached at the third workstation position.

98. The method of claim 94, wherein the wash tray can be moved using the gripper.

99. The method of claim 1, further comprising the step of placing a weight on the slides in the slide tray.

100. The method of claim 93, further comprising the step of using a vacuum device to create a vacuum in the space between the slide and the bath tray.

101. The article of claim 46, further comprising:
a bath tray configured to be placed in a second workstation position, wherein the bath tray comprises a bath well,
wherein the slide tray is configured to be movable to the second workstation position using the gripper, and
wherein the slide tray and the bath tray are configured such that the slide tray can be placed on the bath tray using the gripper and bath liquid can be deposited into the bath well such that the bath liquid contacts the plurality of reaction sites.

102. The article of claim 101, further comprising:
a wash tray configured to be placed in a third workstation position, wherein the wash tray comprises a wash well,
wherein the wash tray is configured to be movable to the third workstation position using the gripper, and
wherein the slide tray and the wash tray are configured such that the slide tray can be placed on the wash tray using the gripper and wash buffer can be deposited into the wash well such that the wash buffer contacts the plurality of reaction sites.

103. The article of claim 102, wherein the liquid dispenser of claim 43 is configured to deposit the wash buffer.

104. The article of claim 102, further comprising a second liquid dispenser that is configured to deposit the wash buffer.

105. The article of claim 102, wherein the wash tray is permanently attached at the third workstation position.

106. The article of claim 102, wherein the wash tray is configured to be movable using the gripper.

107. The article of claim 46, further comprising a weight configured to be placed on the slides in the slide tray.

108. The article of claim 101, further comprising a vacuum device configured to create a vacuum in the space between the slide and the bath tray.
